SEQUENCE LISTING (7 sequences)

SEQ ID NO: 1 - DNA, 20 bp, synthetic construct
  source 1..20: mol_type = other DNA
aggcgctggagaccttacga

SEQ ID NO: 2 - DNA, 20 bp, synthetic construct
  source 1..20: mol_type = other DNA
gtaataacaccagtacggac

SEQ ID NO: 3 - DNA, 20 bp, synthetic construct
  source 1..20: mol_type = other DNA
caggcgacgagtttgaactg

SEQ ID NO: 4 - DNA, 20 bp, synthetic construct
  source 1..20: mol_type = other DNA
ctccgattcagtcccttctg

SEQ ID NO: 5 - DNA, 30 bp, synthetic construct
  source 1..30: mol_type = other DNA
ttttatttgttttttttaaggggttttagt

SEQ ID NO: 6 - DNA, 26 bp, synthetic construct
  source 1..26: mol_type = other DNA
tcctacctataaccataccctaatct

SEQ ID NO: 7 - DNA, 24 bp, synthetic construct
  source 1..24: mol_type = other DNA
aagttttttttattttaaagtttg

(19) United States
(12) Patent Application Publication
(10) Pub. No.: US 2026/0125760 A1
(43) Pub. Date: May 7, 2026

(54) METHODS OF TREATMENT AND DIAGNOSIS

(71) Applicant: Hudson Institute of Medical Research, Clayton, VIC (AU)

(72) Inventors: Ron Firestein, Hawthorn, VIC (AU); Ihara Shazia Adjumain, Oakleigh, VIC (AU); Paul Daniel, Nunawading, VIC (AU)

(21) Appl. No.: 18/940,175

(22) Filed: Nov. 7, 2024

(51) Int. Cl. C12Q 1/6886

(52) U.S. Cl. CPC C12Q 1/6886; C12Q 2600/106; C12Q 2600/154

(57) ABSTRACT

This disclosure relates generally to methods of treating cancers and methods of stratifying subjects having a cancer to a treatment regimen/predicting sensitivity to a treatment regimen, in particular wherein the cancer is detected in a pediatric subject or an adolescent subject.

### REFERENCE TO A SEQUENCE LISTING

This application contains a Sequence Listing which has been submitted electronically in xml format and is hereby incorporated by reference in its entirety. Said xml copy, created on Nov. 6, 2024, is named 35624017-SQL.xml and is 6,955 bytes in size.

### FIELD OF THE ART

This disclosure relates generally to methods of treating cancers and methods of stratifying subjects having a cancer to a treatment regimen/predicting sensitivity to a treatment regimen, in particular wherein the cancer is detected in a paediatric subject or an adolescent subject.

### BACKGROUND

Cancer is a leading cause of death for children and adolescents. Each year, an estimated 400,000 children and adolescents develop cancer. While a good proportion of these cancers can be cured or clinically managed; difficulty of diagnosis, misdiagnosis or delayed diagnosis, incorrect treatment and/or drug toxicity can complicate outcomes. As it is generally not possible to prevent cancer in children and adolescents, the most effective strategy to reduce the burden of cancer in this unique patient population and improve outcomes is to focus on a prompt, correct diagnosis followed by effective, evidence-based therapy with tailored supportive care.

Recent advances in molecular, genetic, and epigenetic profiling have highlighted significant differences in the underlying biology of adult and paediatric cancers, even when they occur in similar tissue types. For example, high-grade gliomas (HGGs), which are malignant central nervous system (CNS) neoplasms can occur in both adult and paediatric populations; however recent studies revealed distinct differences in the epigenetic dysregulation driving oncogenesis in paediatric HGGs (pHGGs) by showing recurrent alterations in histone coding genes H3F3A and HIST1H3B/C genes that are essential for tumorigenesis. In contrast, adult HGGs (aHGGs) preferentially harbor mutations in components of receptor tyrosine kinase (RTK) signaling pathways, such as EGFR and PTEN. Despite apparent similarities, that adult and childhood gliomas have different underlying biologies may necessitate different treatment strategies. For instance, paediatric high-grade gliomas (pHGGs) are well-known to be divergent from adult brain cancers in terms of their genetic complexity, driver mutations, underlying mutational processes, and response to therapy.

Therefore, there remains an urgent need to identify potential biomarkers and drug targets for improved therapies, including for paediatric- and adolescent-onset cancers.

### SUMMARY

In one aspect, there is provided a method of treating a cancer in a subject in whom the cancer is detected at a paediatric or adolescent stage, the method comprising administering to the subject an agent that inhibits MCL1 activity, wherein the cancer detected in the subject at a paediatric or adolescent stage comprises a cell that exhibits DNA hypermethylation at the BCL2L1 gene locus in comparison to the level of DNA methylation at a BCL2L1 gene locus in a non-cancerous cell.

In another aspect, there is provided a method of treating a cancer in a subject, the method comprising:
a) measuring the level of DNA methylation at the BCL2L1 gene locus of a cancer cell obtained from the subject at a paediatric or adolescent stage;
b) identifying whether the cancer cell exhibits DNA hypermethylation at the BCL2L1 gene locus in comparison to the level of DNA methylation at the BCL2L1 gene locus of a non-cancerous cell; and
   where the subject is identified in step (b) as exhibiting DNA hypermethylation at the BCL2L1 gene locus, administering to the subject a therapeutically effective amount of an agent that inhibits MCL1 activity.

In another aspect, there is provided a method of stratifying a subject having a cancer to a treatment regimen comprising an agent that inhibits MCL1 activity, the method comprising:
a) measuring the level of DNA methylation at the BCL2L1 gene locus of a cancer cell obtained from the subject at a paediatric or adolescent stage;
b) identifying whether the cancer cell exhibits DNA hypermethylation at the BCL2L1 gene locus in comparison to the level of DNA methylation level at the BCL2L1 gene locus of a non-cancerous cell; and
   where the subject is identified in step (b) as exhibiting DNA hypermethylation at the BCL2L1 gene locus is stratified to a treatment regimen comprising an agent that inhibits MCL1 activity.

In another aspect, there is provided a method of predicting sensitivity to a treatment regimen comprising an agent that inhibits MCL1 activity in a subject with cancer, the method comprising:
a) measuring the level of DNA methylation at the BCL2L1 gene locus of a cancer cell obtained from the subject at a paediatric or adolescent stage; and
b) identifying whether the cancer cell exhibits DNA hypermethylation at the BCL2L1 gene locus in comparison to the level of DNA methylation level at the BCL2L1 gene locus of a non-cancerous cell;
   wherein hypermethylation at the BCL2L1 gene locus is indicative of sensitivity to a treatment regimen comprising an agent that inhibits MCL1 activity in the subject.

In an embodiment, the BCL2L1 gene locus corresponds to the region spanning chr20:31,664,458 to chr20:31,724,161. In some embodiments, the BCL2L1 gene locus corresponds to the region spanning chr20:31,664,777 to chr20:31,724,161.

In an embodiment, the DNA hypermethylation at the BCL2L1 gene locus is at one or more CpG sites selected from the group consisting of cg06892281, cg25812375, cg17997762, cg17168956, cg15401244, cg14517873, cg21948170, cg00782854, cg12896779, cg07379251, cg21306641, cg03816593, cg11551419, cg23752198, cg11809604, cg07602173, cg09190106, cg04897370, cg14002228, cg00300298, cg08257293, cg02538009, cg00058652, cg11265696, cg18787420, cg12873919 and/or cg13989999.

In an embodiment, the DNA hypermethylation at the BCL2L1 gene locus is at one or more CpG sites within the exon 2 and/or intron 2 junction of BCL2L1 gene.

In an embodiment, the DNA hypermethylation at the BCL2L1 gene locus is at one or more CpG sites within chr20:31,704,843-chr2O:31,721,915. In another embodiment, the DNA hypermethylation at the BCL2L1 gene locus is at one or more CpG sites selected from the group comprising cg13989999, cg12873919, cg18787420, cg00300298, cg08257293, cg00058652, cg11265696, cg04897370, cg14002228 and cg02538009. In another embodiment, the DNA hypermethylation at the BCL2L1 gene locus is at one or more CpG sites selected from the group comprising cg00300298, cg08257293, cg12873919, cg13989999 and cgl8787420. In some embodiments, the DNA hypermethylation at the BCL2L1 gene locus is at cg12873919 and/or cg13989999. In another embodiment, the DNA hypermethylation at the BCL2L1 gene locus is at cg00300298.

In some embodiments, the cancer is a glioma, an Atypical Teratoid Rhabdoid Tumors (ATRT), an ependymoma (EPD), an Ewing sarcoma (ES), an Embryonal Tumor with Multilayered Rosettes (ETMR), a High-Grade Neuroepithelial Tumor (HGNET) or an osteosarcoma (OS). In some embodiments, the cancer is a medulloblastoma, a neuroblastoma, an astrocytoma, a pineoblastoma, a pleomorphic xanthoastrocytoma, a choroid plexus tumour, or a papillary tumour of the pineal region. In an embodiment, the cancer is a glioma. In another embodiment, the cancer is a high-grade glioma.

In an embodiment, the non-cancerous cell is a non-cancerous cell from the same subject.

In an embodiment, the agent that inhibits MCL1 activity is an MCL1-siRNA, an MCL-1 antisense oligonucleotide, S64315, S63845, AZD5991, AMG-176, AMG-397, ABBV-475, ANJ810, TTX-180, ABBV-467 and/or PRT1419. In some embodiments, the agent that inhibits MCL1 activity is AZD5991.

In an embodiment, wherein the cancer is detected in the subject aged between 0-21 years old. In some embodiments, the cancer is detected in the subject aged between 0-19 years old.

In some embodiments, measuring the level of DNA methylation at the BCL2L1 gene locus comprises bisulfite conversion and PCR amplification. In an embodiment, measuring the level of DNA methylation at the BCL2L1 gene locus further comprises pyrosequencing. In another embodiment, measuring the level of DNA methylation at the BCL2L1 gene locus is performed using sodium bisulfite pyrosequencing, methylation-sensitive single nucleotide primer extension (Ms-SNuPE) reaction, methylation-specific PCR or microarray analysis.

In some embodiments, the level of DNA methylation at the BCL2L1 gene locus is measured by the percentage of methylated CpG sites. In some embodiments, hypermethylation is defined by a methylation threshold β-score of at least 0.5. In an embodiment, hypermethylation is defined by a methylation threshold β-score of at least 0.6. In another embodiment, hypermethylation is defined by a methylation threshold β-score of at least 0.7.

In one aspect, there is provided a kit for analysing a biological sample for the presence of DNA methylation at the BCL2L1 gene locus, according to the methods of any one of claims 1-28, the kit comprising
a) a set of nucleic acid primers capable of amplifying the hypermethylated regions of the BCL2L1 gene locus, and/or
b) a set of probes specific for hypermethylated regions of the BCL2L1 gene locus.

The present disclosure also extends an agent that inhibits MCL1 activity for use in treating a cancer in a subject in whom the cancer detected at a paediatric or adolescent stage comprises a cell that exhibits DNA hypermethylation at the BCL2L1 gene locus in comparison to the level of DNA methylation at a BCL2L1 gene locus of a non-cancerous cell.

In some embodiments, the cancer is a glioma, an Atypical Teratoid Rhabdoid Tumors (ATRT), an ependymoma (EPD), an Ewing sarcoma (ES), an Embryonal Tumor with Multilayered Rosettes (ETMR), a High-Grade Neuroepithelial Tumor (HGNET) or an osteosarcoma (OS). In some embodiments, the cancer is a glioma. In an embodiment, the cancer is a high-grade glioma.

In some embodiments, the agent that inhibits MCL1 activity is an MCL1-siRNA, an MCL-1 antisense oligonucleotide, S64315, S63845, AZD5991, AMG-176, AMG-397, ABBV-475, ANJ810, TTX-180, ABBV-467 and/or PRT1419. In an embodiment, the agent that inhibits MCL1 activity is AZD5991.

In an embodiment, the cancer is detected in the subject aged between 0-21 years old. In some embodiments, the cancer is detected in the subject aged between 0-19 years old.

The present disclosure also extends to the use of an agent that inhibits MCL1 activity in the manufacture of a medicament for the treatment of a cancer in a subject in whom the cancer is detected at a paediatric or adolescent stage, wherein the cancer detected in the subject at a paediatric or adolescent stage comprises a cell that exhibits DNA hypermethylation at the BCL2L1 gene locus in comparison to the DNA methylation level at the BCL2L1 gene locus of a non-cancerous cell.

In some embodiments, the agent that inhibits MCL1 activity is an MCL1-siRNA, an MCL-1 antisense oligonucleotide, S64315, S63845, AZD5991, AMG-176, AMG-397, ABBV-475, ANJ810, TTX-180, ABBV-467 and/or PRT1419. In an embodiment, the agent that inhibits MCL1 activity is AZD5991.

In an embodiment, the cancer is detected in the subject is aged between 0-21 years old. In some embodiments, the cancer is detected in the subject is aged between 0-19 years old.

The reference in this specification to any prior publication (or information derived from it), or to any matter which is known, is not, and should not be taken as an acknowledgement or admission or any form of suggestion that the prior publication (or information derived from it) or known matter forms part of the common general knowledge in the field of endeavour to which this specification relates.

Unless specifically defined otherwise, all technical and scientific terms used herein shall be taken to have the same meaning as commonly understood by one of ordinary skill in the art. Although any methods and materials similar or equivalent to those described herein can be used in the practice or testing of the present invention, preferred methods and materials are described. All patents, patent applications, published applications and publications, databases, websites and other published materials referred to throughout the entire disclosure, unless noted otherwise, are incorporated by reference in their entirety. In the event that there is a plurality of definitions for terms, those in this section prevail. Where reference is made to a URL or other such identifier or address, it understood that such identifiers can change and particular information on the internet can come and go, but equivalent information can be found by searching the internet. Reference to the identifier evidences the availability and public dissemination of such information.

### BRIEF DESCRIPTION OF THE DRAWINGS

FIG. 1 shows the results of CRISPR-Cas9 screens identify MCL1 as a key genetic vulnerability in pHGG. (A) Relative enrichment of gene dependencies in paediatric and adult HGGs. An unpaired t-test with multiple comparisons was used to determine statistical significance i.e. FDR<0.05. Δ Mean β-score was calculated as Mean β-score^{(Adult)}-Mean β-score^{(Paediatric)}.

FIG. 2 shows relative viability (to NTC sgRNA) of (A) Paediatric and (B) Adult HGG cell lines after treatment with 2 independent MCL1 targeting sgRNAs. Immunoblot shows MCL1 protein levels under the indicated conditions. Student's t-test. *p<0.0⁵, ***p<0.001, ****p<0.0001, ns=not significant. (C) is a density plot showing IHC score distribution of MCL1 staining in paediatric (blue) and adult (pink) HGG tumor samples.

FIG. 3 shows AZD5991/563845 anti-tumor activity in HGG cell lines. (A) Bar plot represents AUC (normalized to U87-MG) of AZD5991 response in a panel of paediatric (n=28) and adult (n=8) HGG cell lines. (B) Scatterplot showing correlation of MCL1 gene effect (Mean β-scores) of HGG cell lines (n(Adult)=8, n(Paediatric)=27) with AZD5991 drug response (AUC). Pearson's correlation test was used to determine r and significance p. (C) Bar plot represents Log10 IC50 of AZD5991 and S63845 in a panel of paediatric and adult HGG cell lines.

FIG. 4 shows drug-response curves for AZD5991 and S63845 in paediatric HGG cell lines, (A) HGG-080318 and (B) SUPSCG1. Representative graph (n>2 experiments), mean+/−SEM shown. (C) is a bar plot represents normalized caspase 3/7 activation (to control) after treatment with AZD5991 for 24 hours in sensitive and resistant cell lines. Representative graph (n>2 experiments), mean+/−S.E.M shown.

FIG. 5 shows that DNA methylation at the BCL2L1 locus is a potential biomarker of MCL1 inhibitor response in pHGGs. (A) shows hierarchical clustering of correlation coefficients of the 44 CpG sites mapping to the BCL2L1 locus. The CpG sites from top to bottom and left to right are: cg06892281, cg25812375, cg17997762, cg17168956, cg15401244, cg14517873, cg21948170, cg00782854, cg12896779, cg07379251, cg21306641, cg03816593, cg11551419, cg23752198, cg11809604, cg07602173, cg09190106, cg04897370, cg14002228, cg00300298, cg08257293, cg02538009, cg00058652, cg11265696, cg18787420, cg12873919, cg13989999, cg22807314, cg11370496, cg16904055, cg07804698, cg25291404, cg16137862, cg01947399, cg04368572, cg14518385, cg15116802, cg21854837, cg02457826, cg23509889, cg08619561, cg15025647, cg00013684 and cg15488794. A schematic diagram of the location of cluster 1 CpG sites at chr20:ql1.21 is shown. Red dots denote cluster 1 CpG sites (n=238 cells lines). (B) is a waterfall plot showing correlation coefficient of AUC (AZD5991) versus β-score (Methylation) of the 44 BCL2L1 CpG sites. Purple-Cluster 1 CpG sites, Grey-Other CpG sites. (C) is a boxplot showing AZD5991 response of hypermethylated (n=15; β-score>0.5) vs hypomethylated (n=21; β-score<0.5) cell lines at cluster 1 CpG sites. Student's t-test **p<0.01. (D) is a scatterplot showing correlation between methylation status of cluster 1 CpG sites against AZD5991 response in paediatric HGG cell lines. Pearson's correlation test was used to determine r and significance p.

FIG. 6 shows that BCL2L1 is hypermethylated in paediatric CNS tumors. (A) is a scatterplot showing correlation of methylation array β-scores plotted by pyrosequencing values of cell lines. Data points are color-coded (as indicated) based on lineage. Pearson's correlation coefficient, p<0.05. (B) is a density-histogram plot showing distribution of methylation status of cg00300298 obtained via pyrosequencing across fresh patient tissue of pHGGs.

FIG. 7 shows box whisker plots for (A) cell lines and (B) patient samples between cg00300298 methylation status of HGG subtypes and non-malignant brain. Student t-test. *p<0.05, **p<0.01, ****p<0.0001, ns=not significant. (C) is a scatterplot showing correlation of cg00300298 methylation status plotted by BCL2L1 RNA in paediatric HGG. Pearson's correlation coefficient, p<0.05.

FIG. 8 shows (A-B) box plots comparing the distribution of cg00300298 methylation status between paediatric and adult HGG subtypes in (A) cell lines and (B) patient samples. Student t-test, *p<0.05, ns=not significant. (C) is a scatterplot showing correlation between ABALON RNA levels vs methylation status of cg00300298 in pediatric HGGs. Pearson's correlation coefficient, p=ns. (D) is a scatterplot showing correlation between BCL2L1 protein levels vs methylation status of cg00300298 in pediatric HGGs. Pearson's correlation coefficient, p<0.05.

FIG. 9 shows (A) immunoblots of BCL2L1 protein levels under indicated conditions; and drug-response curves for AZD5991 following BCL2L1 KO by two independent sgRNA and control-NTC in indicated cell lines. Representative graph shown (n=3). Mean+/−SD. (B) shows immunoblots of BCL2L1 protein levels under indicated conditions. Drug-response curves for AZD5991 following 2 weeks post sgRNA transduction of exogenous BCL2L1 and control-empty vector in indicated cell lines. Representative graph shown (n=3). Mean+/−SD.

FIG. 10 shows that Cluster 1 methylation is a predictor of MCL1 inhibitor response in paediatric CNS and non-CNS cancers. (A) Boxplots illustrating the distribution of cluster 1CpGsites (cg12873919 and cg13989999) methylation status across paediatric and adult cancers, derived from DepMap datasets. ***p<0.0001. (B) Violin plots depicting the distribution of cg00300298 methylation status across various paediatric cancers and non-malignant brain cell lines. (C) Violin plots depicting the distribution of cg00300298 methylation status across various paediatric cancers and non-malignant brain patient tissue samples. (D) Stacked bar plot represents the % of methylated vs unmethylated paediatric cancer cell lines based on a β-score>0.7.

FIG. 11 shows boxplots illustrating the methylation patterns of the individual CpG sites (cg00300298, cg08257293, cg12873919, cg13989999, and cg18787420 across and adult and paediatric non-malignant brain tissue, derived from an external dataset (34). Student t-test. *p<0.05, **p<0.01, ***p<0.001, ****p<0.0001, ns=not significant.

FIG. 12 shows boxplots illustrating the mean distribution of (A) cluster 1 CpG sites methylation status across non-malignant paediatric (n=36) and adult (n=182) brain tissue, derived from an external dataset. Age cut-off to define adult and paediatric=21, Student's t-test ***p<0.0001. (B) Boxplots illustrating the median global methylation patterns across non-malignant paediatric (n=36) and adult (n=182) brain tissue, derived from an external dataset (34). Age cut-off=21, Student's t-test ***p<0.0001.

FIG. 13 shows BCL2L1 hypermethylation predicts MCL1 dependency in an orthotopic CNS mouse model. Drug-response curves for (A) AZD5991 and (B) S63845 in ATRT, OS (Osteosarcoma) and EPD (Ependymoma) groups based on BCL2L1 methylation as color coded; methylated (red) or unmethylated (green). Representative graph (n≥2 experiments), mean+/−S.E.M shown, Extra sum-of-squares F test p<0.0001.

FIG. 14 shows (A) a schematic of in vivo-CRISPR-Cas9 screen utilizing orthotopic xenograft models of ATRT. (B) Genes ranked by gene dependency scores (0-score) for BT12 and BT16 orthotopic in vivo screens.

FIG. 15 shows a schematic representation illustrating the effect of BCL2L1 methylation on MCL1 inhibitor response in malignant and normal tissue in the setting of paediatric and adult cancer patients.

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described.

For the purposes of the present invention, the following definitions included in this section are general, broad definitions. Additional terms for the purpose of describing particular aspects and embodiments of the invention are provided at the appropriate location in the detailed description, and are not intended to limit the scope of the invention.

All sequence identifiers (e.g., GenBank ID, Addgene reference, EMBL-Bank ID, genome assembly, Illumina Methylation Array etc.) provided herein were current at the filing date.

The articles “a” and “an” are used herein to refer to one or to more than one (i.e. to at least one) of the grammatical object of the article. By way of example, “a biomarker” means one biomarker or more than one biomarker, unless otherwise indicated.

As used herein, “and/or” refers to and encompasses any and all possible combinations of one or more of the associated listed items, as well as the lack of combinations when interpreted in the alternative (or).

As used herein, the term “about” refers to a quantity, level, value, number, dimension, size, percentage or amount that varies by as much as 10% (e.g., by 10%, 9%, 8%, 7%, 6%, 5%, 4%, 3%, 2% or 1%) to a reference quantity, level, value, number, dimension, size, percentage or amount.

The present description uses numerical ranges to quantify certain parameters relating to this disclosure. It should be understood that when numerical ranges are provided, such ranges are to be construed as providing support for claim limitations that recite the lower value of the range as well as claim limitations that recite the upper value of the range. For example, a disclosed numerical range of 10 to 100 provides support for a claim reciting “greater than 10” (with no upper bounds) and a claim reciting “less than 100” (with no lower bounds) and provided support for and includes the end points of 10 and 100.

Throughout this specification, unless the context requires otherwise, the words “comprise”, “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands.

The term “corresponding” as used herein in reference to a particular gene or nucleic acid sequence is intended to mean an analogous or equivalent or comparable gene or nucleic acid sequence. For example, where reference is made to a corresponding endogenous gene, it is intended to mean the analogous, equivalent or comparable naturally-occurring gene. Where reference is made to a corresponding exogenous gene, it is intended to mean an analogous, equivalent or comparable exogenous gene. Where reference is made to a corresponding wild type equivalent of a nucleic acid sequence, it is intended to mean an analogous or comparable exogenous gene. Where reference is made to a corresponding equivalent of a nucleic acid sequence in a non-cancerous cell, it is intended to mean a comparable nucleic acid sequence in a cell that is not cancerous, or has not been derived from a cancer or tumour tissue. In some embodiments, the corresponding gene or nucleic acid sequence has analogous or equivalent function or having sequence similarity. In an embodiment, the corresponding gene or nucleic acid sequence may be identical in function and/or sequence. In another embodiment, the corresponding gene or nucleic acid sequence may have about the same function or activity. In another embodiment, the corresponding gene or nucleic acid sequence may have reduced function or activity. In some embodiments, the phrase “corresponds to” or “corresponding to” refers to a nucleic acid sequence that displays substantial sequence identity to a reference nucleic acid sequence. In general the nucleic acid sequence will display at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or even up to 100% sequence identity to the reference nucleic acid sequence.

As used herein, the terms “encode”, “encoding” and the like refer to the capacity of a nucleic acid to provide for another nucleic acid or a polypeptide or protein. For example, a nucleic acid sequence is said to “encode” a polypeptide if it can be transcribed and/or translated to produce the polypeptide or if it can be processed into a form that can be transcribed and/or translated to produce the polypeptide. Such a nucleic acid sequence may include a coding sequence or both a coding sequence and a non-coding sequence. Thus, the terms “encode”, “encoding” and the like include an RNA product resulting from transcription of a DNA molecule, a protein resulting from translation of an RNA molecule, a protein resulting from transcription of a DNA molecule to form an RNA product and the subsequent translation of the RNA product, or a protein resulting from transcription of a DNA molecule to provide an RNA product, processing of the RNA product to provide a processed RNA product (e.g., mRNA) and the subsequent translation of the processed RNA product.

The term “expression”, as used herein, typically refers to any step involved in the production of an RNA molecule or a polypeptide, such as by transcription, post-transcriptional modification, translation, post-translational modification, and secretion. The term “expressed” or “expression” as used herein, typically refers to any step involved in the production of an RNA molecule or a polypeptide, such as by transcription, post-transcriptional modification, translation, post-translational modification, and secretion.

The term “gene” is used herein to refer to a unit of inheritance that comprises a coding sequence and optionally transcriptional and/or translational regulatory sequences and/or non-translated sequences (i.e., introns, 5′ and 3′ untranslated sequences) whether or not such regulatory sequences are adjacent to coding and/or transcribed sequences. Accordingly, a gene may include or encode promoter sequences, signal peptides, terminators, translational regulatory sequences such as ribosome binding sites and internal ribosome entry sites, enhancers, silencers, insulators, boundary elements, replication origins, matrix attachment sites, and locus control regions. In some embodiments the gene may comprise only coding sequence. In other embodiments, the gene may comprise coding sequences and non-coding sequences.

By “isolated” is meant material that is substantially or essentially removed from components that normally accompany it in its native state.

The terms “control”, “control sample”, “reference”, “reference sample”, “normal”, and “normal sample” can be used to describe a sample or a corresponding genomic region from a subject that does not have a particular condition, or is otherwise healthy. Alternatively, the control, control sample, reference, reference sample, normal or normal sample can be used to describe a sample or a corresponding genomic region in un-diseased tissue or an un-diseased cell (i.e. that does not have a particular condition). In an example, a method as disclosed herein can be performed on a subject having a tumor, where the reference sample is a sample taken from a healthy tissue of the subject. A reference sample can be obtained from the subject, or from a database. The reference can be, e.g., a reference genome that is used to map sequence reads obtained from sequencing a sample from the subject. A reference genome can refer to a genome to which sequence reads from the biological sample and a constitutional sample can be aligned and compared. In another example, the control can be cells from non-malignant tissue.

The phrase “healthy,” as used herein, can refer to a subject possessing good health. A healthy subject can demonstrate an absence of any malignant or non-malignant disease. A “healthy individual” can have other diseases or conditions, unrelated to the condition being assayed, which can normally not be considered “healthy.”

The term “sample,” “biological sample” or “patient sample” can include any cell, tissue or material derived from a living or dead subject. A biological sample can be a cell-free sample. A biological sample can comprise a nucleic acid (e.g., DNA or RNA) or a fragment thereof. The term “nucleic acid” can refer to deoxyribonucleic acid (DNA), ribonucleic acid (RNA) or any hybrid or fragment thereof. The nucleic acid in the sample can be a cell-free nucleic acid. A sample can be a liquid sample or a solid sample (e.g., a cell or tissue sample). The technology is not limited to particular sample types. A biological sample can be a bodily fluid, such as blood, plasma, serum, urine. For example, in some embodiments the sample is a tissue sample, a blood sample, a serum sample, or a sputum sample. In certain embodiments a tissue sample comprises cancer or tumour tissue. A biological sample can be treated to physically disrupt tissue or cell structure (e.g., centrifugation and/or cell lysis), thus releasing intracellular components into a solution which can further contain enzymes, buffers, salts, detergents, and the like which can be used to prepare the sample for analysis.

The terms “treating”, “treatment” and the like, are used interchangeably herein to mean relieving, reducing, alleviating, ameliorating or otherwise inhibiting the condition, including one or more symptoms of the condition. It is also to be understood that terms “treating”, “treatment” and the like do not imply that the condition, or a symptom thereof, is permanently relieved, reduced, alleviated, ameliorated or otherwise inhibited and therefore also encompasses the temporary relief, reduction, alleviation, amelioration or otherwise inhibition of the condition, or of a symptom thereof. The term “treating” as used herein, unless otherwise indicated, means alleviating, inhibiting the progress of, or preventing, either partially or completely, the pathophysiology of cancer, or a symptom thereof.

A “therapeutically-effective” amount as used herein is an amount, at dosages and for periods of time necessary, that is sufficient to alleviate (e.g., mitigate, decrease, reduce) at least one of the symptoms associated with a disease state, to achieve the desired therapeutic result. Alternatively stated, a “therapeutically-effective” amount is an amount that is sufficient to provide some improvement in the condition of the subject. A therapeutically effective amount of the agent may vary according to factors such as the disease state, age, sex, and weight of the individual, and the ability of the agent to elicit a desired response in the individual. A therapeutically effective amount is also one in which any toxic or detrimental effects of the agent is outweighed by the therapeutically beneficial effects. A “therapeutically effective amount” will fall in a relatively broad range that can be determined through experimentation and/or clinical trials. Other effective dosages can be readily established by one of ordinary skill in the art through routine trials establishing dose response curves. In some examples, more than one administration (e.g., two, three, four or more administrations) may be employed to achieve the desired level of gene expression over a period of various intervals, e.g., daily, weekly, monthly, yearly, etc.

### 2. DNA Methylation

The present disclosure is predicated, at least in part, on the inventors' surprising finding that hypermethylation of a BCL21 gene locus in a cancer in a subject in whom the cancer is detected at a paediatric or adolescent stage is capable of predicting sensitivity of the cancer to MCL1 inhibition.

Typically, DNA methylation is an epigenetic mechanism that involves the transfer of a methyl group onto the C5 position of the cytosine to form 5-methylcytosine, wherein the cytosine is usually occurring in a dinucleotide sequence including an adjacent guanine, where the cytosine is located 5′ of the guanine. This is referred to as a “CpG site” or “CpG dinucleotide sequence”.

As used herein, a “CpG island” refers to a G:C-rich region of genomic DNA containing an increased number of CpG dinucleotides relative to total genomic DNA. A CpG island can be at least 100, 200, or more base pairs in length, where the G:C content of the region is at least 50% and the ratio of observed CpG frequency over expected frequency is 0.6; in some instances, a CpG island can be at least 500 base pairs in length, where the G:C content of the region is at least 55%) and the ratio of observed CpG frequency over expected frequency is 0.65. The observed CpG frequency over expected frequency can be calculated according to the method provided in Gardiner-Garden et al (1987) J. Mol. Biol. 196: 261-281. However, it will be appreciated though that other sequences in the human genome can also be methylated, such as CpA and CpT (see Ramsahoye (2000) Proc. Natl. Acad. Sci. USA 97: 5237-5242; Salmon and Kaye (1970) Biochim. Biophys. Acta. 204: 340-351; Grafstrom (1985) Nucleic Acids Res. 13: 2827-2842; Nyce (1986) Nucleic Acids Res. 14: 4353-4367; Woodcock (1987) Biochem. Biophys. Res. Commun. 145: 888-894).

As used herein, a “methylated nucleic acid molecule” refers to a nucleic acid molecule that contains one or more methylated nucleotides. The terms “methylated DNA” or a “methylated nucleotide” or “hypermethylation” or “hypermethylated nucleotide” or refers to the presence of a methyl moiety on one or more nucleotide bases of a genomic region or genetic sequence of interest, wherein the methyl moiety is not present in the corresponding nucleotide base/s of the corresponding genomic region or genetic sequence in a comparator sample, tissue or cell; which maybe a control, control sample, reference, reference sample, normal or normal sample, tissue or cell. It must be noted that in vitro amplified DNA is usually unmethylated because typical in vitro DNA amplification methods do not retain the methylation pattern of the amplification template. However, “unmethylated DNA” or “methylated DNA” can also refer to amplified DNA whose original template was unmethylated or methylated, respectively.

The level of DNA methylation can refer to, or indicate the methylation of a particular CpG site. The level of DNA methylation can also refer to, or indicate the methylation state of every base in the sequence or it can indicate the methylation state of a subset of the bases (e.g., of one or more cytosines) within the sequence, or can indicate information regarding regional methylation density within the sequence with or without providing precise information of the locations within the sequence the methylation occurs. The “level of DNA methylation” or the “methylation state” or “methylation status” or “methylation profile”, which are used herein interchangeable, can be represented or indicated by a “methylation score” or “methylation value” which can refer to a methylation frequency, fraction, ratio, percentage, etc. As used herein, “methylation frequency” or “methylation percent (%)” refer to the number of instances in which a molecule or locus is methylated relative to the number of instances the molecule or locus is unmethylated. In an embodiment, the level of DNA methylation is measured by the percentage of methylated CpG sites.

The terms “methylation state”, “methylation profile”, and “methylation status” also refer to the relative concentration, absolute concentration, or pattern of methylated C or unmethylated C for a particular CpG site, genomic site, or throughout any particular region of a nucleic acid in a biological sample. For example, if the cytosine (C) residue(s) within a nucleic acid sequence are methylated it may be referred to as “hypermethylated” or having “increased methylation”, whereas if the cytosine (C) residue(s) within a DNA sequence are not methylated it may be referred to as “hypomethylated” or having “decreased methylation”. Likewise, if the cytosine (C) residue(s) within a nucleic acid sequence are methylated as compared to another nucleic acid sequence (e.g., from a different region or from a different individual, etc.) that sequence is considered hypermethylated or having increased methylation compared to the other nucleic acid sequence. Alternatively, if the cytosine (C) residue(s) within a DNA sequence are not methylated as compared to another nucleic acid sequence (e.g., from a different region or from a different individual, etc.) that sequence is considered hypomethylated or having decreased methylation compared to the other nucleic acid sequence. Additionally, the terms “methylation state”, “methylation profile”, and “methylation status” as used herein can be used to describe the methylation of a single CpG site, or a collective of CpG sites across a region of a nucleic acid. It can also be appreciated that two nucleic acids may have the same or similar methylation frequency or methylation percent may have different methylation patterns when the number of methylated and unmethylated nucleotides are the same or similar throughout the region but the locations of methylated and unmethylated nucleotides are different. Sequences are said to be “differentially methylated” or as having a “difference in methylation” or having a “different methylation state” when they differ in the extent (e.g., one has increased or decreased methylation relative to the other), frequency, or pattern of methylation. The term “differential methylation” refers to a difference in the level or pattern of nucleic acid methylation in a cancer positive sample as compared with the level or pattern of nucleic acid methylation in a cancer negative sample.

Methylation state frequency can be used to describe a population of individuals or a sample from a single individual. For example, a nucleotide locus having a methylation state frequency of 50% is methylated in 50% of instances and unmethylated in 50% of instances. Such a frequency can be used, for example, to describe the degree to which a CpG site, a nucleotide locus or nucleic acid region is methylated in a population of individuals or a collection of nucleic acids. Thus, when methylation in a first population or pool of nucleic acid molecules is different from methylation in a second population or pool of nucleic acid molecules, the methylation state frequency of the first population or pool will be different from the methylation state frequency of the second population or pool. Such a frequency also can be used, for example, to describe the degree to which a nucleotide locus or nucleic acid region is methylated in a single individual. For example, such a frequency can be used to describe the degree to which a group of cells from a tissue sample are methylated or unmethylated at a nucleotide locus or nucleic acid region.

A methylation score can be generated, for example, by quantifying the amount of intact nucleic acid present following restriction digestion with a methylation dependent restriction enzyme or by comparing amplification profiles or probe binding profiles after bisulfite reaction or by comparing sequences of bisulfite-treated and untreated nucleic acids. Accordingly, a value, e.g., a methylation score, represents the methylation status and can thus be used as a quantitative indicator of methylation status. Examples of methylation scores commonly used in the art (particular where DNA methylation is assayed using a methylation array) to determine or measure the methylation status or level of methylation of a loci of interest, include β-value (sometimes also referred to as β-score) and M value. The M-value is calculated as the log 2 ratio of the intensities of methylated probe versus unmethylated probe. In an embodiment, the level of DNA methylation is measured using M-values. The β-value is the ratio of the methylated probe intensity and the overall intensity (sum of methylated and unmethylated probe intensities). β-values range from 0 to 1 and can represent the proportion of how many cells had a methylated base for that probe site. In another embodiment, the level of DNA methylation is measured using β-values. In some examples, a region is considered hypomethylated when it has a β-score<0.5; and a region is considered hypermethylated when it has a β-score>5. In an embodiment, DNA hypermethylation is defined by a methylation threshold β-score of at least 0.5. In another embodiment, hypermethylation is defined by a methylation threshold β-score of at least 0.6. In another embodiment, hypermethylation is defined by a methylation threshold β-score of at least 0.7.

Suitable assays and methods for measuring DNA methylation will be known to the person skilled in the art. The most frequently used method for analyzing a nucleic acid for the presence of 5-methylcytosine is based upon the bisulfite method described by Frommer, et al. for the detection of 5-methylcytosines in DNA (Frommer et al. (1992) Proc. Natl. Acad. Sci. USA 89: 1827-31 explicitly incorporated herein by reference in its entirety for all purposes) or variations thereof.

The bisulfite method of mapping 5-methylcytosines is made possible because cytosine, but not 5-methylcytosine, reacts with hydrogen sulfite ion (also known as bisulfite). The reaction is usually performed according to the following steps: first, cytosine reacts with a bisulfite reagent (e.g. hydrogen sulfite) to form a sulfonated cytosine. Deamination of the sulfonated reaction intermediate results in a sulfonated uracil, which is desulfonated under alkaline conditions to form uracil. Detection is possible because uracil base pairs with adenine (thus behaving like thymine), whereas 5-methylcytosine base pairs with guanine (thus behaving like cytosine). This makes the discrimination of methylated cytosines from non-methylated cytosines possible by, e.g., bisulfite genomic sequencing, methylation-specific PCR (MSP) or using an assay comprising sequence-specific probe cleavage, e.g., a QuARTS flap endonuclease assay.

Other related technologies for detecting DNA methylation comprise enclosing the DNA of interest in an agarose matrix to deter diffusion and renaturation of the DNA, as bisulfite only reacts with single-stranded DNA, and replacing precipitation and purification steps with a fast dialysis (Olek A, et al. (1996) “A modified and improved method for bisulfite based cytosine methylation analysis” Nucleic Acids Res. 24: 5064-6). It is thus possible to analyze individual cells for methylation status. Overview of conventional methods for detecting DNA is provided by Rein, T., et al. (1998) Nucleic Acids Res. 26: 2255; and Zhang et al. (2024) ACS Sensors.

The bisulfite technique typically involves amplifying short, specific fragments of a known nucleic acid subsequent to a bisulfite treatment, then either assaying the product by sequencing or a primer extension reaction (Gonzalgo & Jones (1997) Nucleic Acids Res. 25: 2529-31; WO 95/00669; U.S. Pat. No. 6,251,594) to analyze individual cytosine positions. Some methods use enzymatic digestion (Xiong & Laird (1997) Nucleic Acids Res. 25: 2532-4). Detection by hybridization has also been described in the art (Olek et al., WO 99/28498). Additionally, use of the bisulfite technique for methylation detection with respect to individual genes has been described (Grigg & Clark (1994) Bioessays 16: 431-6; Zeschnigk et al. (1997) Hum Mol Genet. 6: 387-95; Feil et al. (1994) Nucleic Acids Res. 22: 695; Martin et al. (1995) Gene 157: 261-4; WO 9746705; WO 9515373).

Various methylation assay procedures can be used in conjunction with bisulfite treatment according to the present technology. These assays allow for determination of the methylation state of one or a plurality of CpG sites within a nucleic acid sequence. Such assays involve, among other techniques, sequencing of bisulfite-treated nucleic acid, PCR (for sequence-specific amplification), Southern blot analysis, and use of methylation-sensitive restriction enzymes. Another common method of measuring DNA methylation is methylation arrays; and the most commonly used arrays are manufactured by Illumina.

In another example, a quantitative methylation assay useful for determining DNA methylation levels at specific loci in small amounts of genomic DNA; methylation-dependent sequence differences are first introduced into the genomic DNA by standard bisulfite treatment. PCR amplification of the bisulfite converted DNA is then performed using primers specific for the CpG sites/regions of interest, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. This technique can be used on DNA obtained from microdissected paraffin-embedded tissue samples.

In an embodiment, the level of DNA methylation is measured using bisulfite conversion and PCR amplification. In an embodiment, the level of DNA methylation is measured using pyrosequencing. In an embodiment, the level of DNA methylation is measured using sodium bisulfite pyrosequencing, methylation-sensitive single nucleotide primer extension (Ms-SNuPE) reaction, methylation-specific PCR or microarray analysis.

### 3. BCL2L1

The present disclosure is predicated, at least in part, on the inventors′ surprising finding that hypermethylation of a BCL21 gene locus in a cancer in a subject in whom the cancer is detected at a paediatric or adolescent stage is capable of predicting sensitivity of the cancer to MCL1 inhibition.

The BCL21L1 gene encodes the Bcl-2-like protein 1, and belongs to the Bcl-2 protein family. Bcl-2 family members form hetero- or homodimers and act as anti- or pro-apoptotic regulators that are involved in a wide variety of cellular activities. Importantly, Bcl-2 family proteins function to control apoptosis by governing mitochondrial outer membrane permeabilisation, a key step in the intrinsic pathway of apoptosis. Members of the BCL-2 family regulate apoptosis in mammals and other animals. Their molecular structure and function, as well as their protein dynamics, are highly conserved.

BCL2L1 has been reported to inhibit activation of caspases, and have important roles in the regulation of apoptosis by blocking the voltage-dependent anion channel (VDAC) by binding to it and preventing the release of the caspase activator, CYC1, from the mitochondrial membrane. Other roles BCL2L1 has been reporting in having include regulating the G2 checkpoint and progression to cytokinesis during mitosis.

In an embodiment, the human BCL21L1 gene corresponds to Entrez gene ID: 598, locus reference number: NG_029002, and NCBI Reference Sequence: NG_029002.1, and spans the genomic region: hg38 chr20:31,664,458-31,722,868. In an embodiment, the human Bcl-2-like protein 1 corresponds to UniProtKB/Swiss-Prot: Q07817. BCL2L1 is also known as BCLX; BCL2L; Bcl-X; PPP1R52; and BCL-XL/S. Alternative splicing of BCL21L1 transcripts give rise to human proteins Bcl-xL and Bcl-xS. The longer isoform (Bcl-xL) acts as an apoptotic inhibitor and the shorter form (Bcl-xS) acts as an apoptotic activator.

Evasion of apoptosis is a hallmark of cancer and therefore it is not surprising that BCL-2 family of proteins play a key role in tumor formation and survival. BCL2L1 has previously been implicated in cancer. Mutations, genetic rearrangements, aberrant expression and copy number variation in BCL2L1 have been detected in human cancers and implicated in cancer progression (see Hata et al. (2015) Cancer Discov 5:475-487 and Beroukhim et al. (2010) Nature 463: 899-905). A negative correlation between mRNA expression and promoter DNA methylation of BCL2L1 was reported in gynaecologic cancers, however it must also be noted that there no was correlation observed for mRNA expression and survival for BCL2L1 (Wang et al. (2020) Cell Proliferation 53: e12826). Further, there has been mixed reports on the utility of BCL21 copy number variation and protein levels (including in combination with other biomarkers) in predicting sensitivity to certain compounds (Cerella et al. (2024) Leukemia 38:67-81 and Wei et al. (2023) Anim Models Exp Med. 6:245-254).

However, to date, differential methylation of BCL2L1 gene has not been linked to sensitivity to any specific anti-cancer therapeutics. The inventors have surprisingly shown for the first time, that BCL2L1 methylation predicts sensitivity to MCL1 inhibitor response in paediatric and adolescent cancers, but not in the corresponding adult cancers. That is, the inventors have discovered that cancers that exhibit DNA hypermethylation at the BCL2L1 gene locus in comparison to the level of DNA methylation at a BCL2L1 gene locus in a non-cancerous cell is responsive to treatment with an agent that inhibits MCL1 activity.

In one embodiment, the BCL2L1 gene locus corresponds to the region spanning chr20:31,664,458 to chr20:31,724,161. In another embodiment, the BCL2L1 gene locus corresponds to exon 2 and/or intron 2 junction of the BCL2L1 gene. In another embodiment, the BCL2L1 gene locus corresponds to exon 2 and intron 2 junction of the BCL2L1 gene. In another embodiment, the BCL2L1 gene locus corresponds to exon 2 or intron 2 junction of the BCL2L1 gene. In another embodiment, the BCL2L1 gene locus corresponds to exon 2 of the BCL2L1 gene. In another embodiment, the BCL2L1 gene locus corresponds the intron 2 junction of the BCL2L1 gene.

In one embodiment, the DNA hypermethylation at the BCL2L1 gene locus is at one or more CpG sites selected from the group consisting of cg06892281, cg25812375, cg17997762, cg17168956, cg15401244, cg14517873, cg21948170, cg00782854, cg12896779, cg07379251, cg21306641, cg03816593, cg11551419, cg23752198, cg11809604, cg07602173, cg09190106, cg04897370, cg14002228, cg00300298, cg08257293, cg02538009, cg00058652, cg11265696, cg18787420, cg12873919 and cg13989999. The corresponding genomic coordinates for the CpG sites are provided in Table 4.

In an embodiment, the DNA hypermethylation at the BCL2L1 gene locus is at one or more CpG sites at chr20:31695147-31695148, chr20:31700232-31700233, chr20:31686061-31686062, chr20:31709303-31709304, chr20:31680723-31680724, chr20:31706215-31706216, chr20:31671139-31671140, chr20:31723055-31723056, chr20:31723263-31723264, chr20:31722945-31722946, chr20:31722683-31722684, chr20:31723900-31723901, chr20:31723436-31723437, chr20:31724160-31724161, chr20:31724044-31724045, chr20:31722627-31722628, chr20:31722606-31722607, chr20:31713376-31713377, chr20:31713169-31713170, chr20:31721153-31721154, chr20:31720637-31720638, chr20:31704843-31704844, chr20:31720457-31720458, chr20:31717824-31717825, chr20:31721663-31721664, chr20:31721824-31721825, and/or chr20:31721914-31721915.

In another embodiment, the DNA hypermethylation at the BCL2L1 gene locus is at one or more CpG sites found within the exon 2 and/or intron 2 junction of the BCL2L1 gene. In another embodiment, the DNA hypermethylation at the BCL2L1 gene locus is at one or more CpG sites within chr20:31,704,843-chr20:31,721,915.

In some embodiments, the DNA hypermethylation at the BCL2L1 gene locus is at one or more CpG sites selected from the group comprising cg13989999, cg12873919, cg18787420, cg00300298, cg08257293, cg00058652, cg11265696, cg04897370, cg14002228 and cg02538009. The corresponding genomic coordinates for the CpG sites are provided in Tables 4-5. In some embodiment, the DNA hypermethylation at the BCL2L1 gene locus is at one or more CpG sites at chr20:31721914-31721915, chr20:31721824-31721825, chr20:31721663-31721664, chr20:31721153-31721154, chr20:31720637-31720638, chr20:31720457-31720458, chr20:31717824-31717825, chr20:31713376-31713377, chr20:31713169-31713170 and/or chr20:31704843-31704844.

In some embodiments, the DNA hypermethylation at the BCL2L1 gene locus is at one or more CpG sites selected from the group comprising cg00300298, cg08257293, cg12873919, cg13989999 and cg18787420. In some embodiment, the DNA hypermethylation at the BCL2L1 gene locus is at one or more CpG sites at chr20:31721153-31721154, chr20:31720637-31720638, chr20:31721824-31721825, chr20:31721914-31721915 and/or chr20:31721663-31721664.

In some embodiments, the DNA hypermethylation at the BCL2L1 gene locus is at cg12873919 and/or cg13989999. In some embodiments, the DNA hypermethylation at the BCL2L1 gene locus is at chr20:31721824-31721825 and/or chr20:31721914-31721915.

In yet another embodiment, the DNA hypermethylation at the BCL2L1 gene locus is at cg00300298. In another embodiment, the DNA hypermethylation at the BCL2L1 gene locus is at chr20:31721153-31721154.

Hypermethylation of any individual CpG sites described herein, or one or more CpG sites in BCL2L1 gene locus of a cancer cell when compared to the level of DNA methylation at the corresponding CpG site/s at a BCL2L1 gene locus in a non-cancerous cell, is indicative of sensitivity of the cancer to MCL1 inhibition therapy. In contrast, hypomethylation or lack of methylation of any individual CpG sites described herein, or one or more CpG sites in BCL2L1 gene locus, of a cancer cell, is indicative of a cancer that is not sensitive to MCL1 inhibition therapy.

Purely by the way of illustrative example, for the methods disclosed herein, hypermethylation of any one of the CpG sites selected from the group consisting of cg13989999, cg12873919, cg18787420, cg00300298, cg08257293, cg00058652, cg11265696, cg04897370, cg14002228 and cg02538009, is indicative of a cancer that is sensitive to MCL1 inhibition therapy. For example, a β-value or β-score of ≥0.5 for the relevant CpG site would be indicative of a cancer that is sensitive to MCL1 inhibition therapy. A β-value or β-score of <0.5 for the relevant CpG site would be indicative of a cancer that is not sensitive to MCL1 inhibition therapy.

The skilled person would also understand from the present disclosure that an increased level of methylation (e.g. hypermethylation) across a region of exon 2 and/or intron 2 junction of the BCL2L1 gene or chr20:31,704,843-chr2O:31,721,915 as described herein, (i.e. an increased methylation frequency, concentration or percentage of methylated CpG sites in the region) in a cancer cell, in comparison to the level of DNA methylation at the corresponding region in a non-cancerous cell, is indicative of a cancer that is sensitive to MCL1 inhibition therapy.

### 4. Treatment of Cancer and Methods of Stratifying Patients to Treatment Regimens

The methods of the present disclosure generally relate to methods of treating a cancer or a tumor in a subject; methods of stratifying a subject having a cancer to a particular treatment regimen; and methods of predicting sensitivity to MCL1 inhibition treatment in a subject with cancer.

As disclosed herein, the methods of the present disclosure relate to treating a cancer in a subject in whom the cancer is detected at a paediatric or adolescent stage, the method comprising administering to the subject an agent that inhibits MCL1 activity, wherein the cancer detected in the subject at a paediatric or adolescent stage comprises a cell that exhibits DNA hypermethylation at the BCL2L1 gene locus in comparison to the level of DNA methylation at a BCL2L1 gene locus in a non-cancerous cell. Also disclosed herein are methods of treating a cancer in a subject, the method comprising measuring the level of DNA methylation at the BCL2L1 gene locus of a cancer cell obtained from the subject at a paediatric or adolescent stage; identifying whether the cancer cell exhibits DNA hypermethylation at the BCL2L1 gene locus in comparison to the level of DNA methylation at the BCL2L1 gene locus of a non-cancerous cell; where the subject identified as exhibiting DNA hypermethylation at the BCL2L1 gene locus is administered a therapeutically effective amount of an agent that inhibits MCL1 activity. Further, disclosed herein are methods of stratifying a subject having a cancer to a treatment regimen comprising an agent that inhibits MCL1 activity, the method comprising measuring the level of DNA methylation at the BCL2L1 gene locus of a cancer cell obtained from the subject at a paediatric or adolescent stage; identifying whether the cancer cell exhibits DNA hypermethylation at the BCL2L1 gene locus in comparison to the level of DNA methylation level at the BCL2L1 gene locus of a non-cancerous cell; and wherein the subject is identified as exhibiting DNA hypermethylation at the BCL2L1 gene locus is stratified to a treatment regimen comprising an agent that inhibits MCL1 activity. In another aspect of the present disclosure, there is provided a method of predicting sensitivity to a treatment regimen comprising an agent that inhibits MCL1 activity in a subject with cancer, the method comprising measuring the level of DNA methylation at the BCL2L1 gene locus of a cancer cell obtained from the subject at a paediatric or adolescent stage; and identifying whether the cancer cell exhibits DNA hypermethylation at the BCL2L1 gene locus in comparison to the level of DNA methylation level at the BCL2L1 gene locus of a non-cancerous cell; wherein hypermethylation at the BCL2L1 gene locus is indicative of sensitivity to a treatment regimen comprising an agent that inhibits MCL1 activity in the subject.

The subject can be any human patient, such as a cancer patient, or a patient suspected of having a cancer. In some embodiments, the subject has been diagnosed as having a cancer or a tumour. In some cases, the subject has not begun treatment. In some cases, the subject has commenced treatment. In some cases, the subject is in a particular stage of cancer treatment.

The terms “subject”, “individual” and “patient” are used interchangeably herein. Where relevant in the description herein, the subject can have any type of cancer or tumor. In an embodiment, the subject is a paediatric subject or an adolescent subject. In an embodiment, the subject is a paediatric subject. In an embodiment, the subject is an adolescent subject.

Paediatrics and adolescent medicine are specialised branches of medical care that take into account the substantial physiological; developmental, metabolic and biochemical differences between infants, children, maturing individuals; and that of a fully matured adult. Aside from the body size differences that are typically observed across subsets of patients, many pediatric, adolescent and young adult patients are still physically developing and may have different growth and developmental issues (e.g. hormonal development) that are not typically present in adult patients. Paediatrics and adolescent medicine is generally understood to refer to the medical care of infants, children, adolescents, and young adults aged 24 or younger (see Sawyer et al. (2018) The Lancet Child & Adolescent Health 2:223-228). The World Health Organisation defines adolescence as a period of development up to 19 years of age. The American Academy of Pediatrics recommends that pediatric care cover patients through to the age of 21. The different ranges for the age cut off for paediatric and adolescent medicine is likely due to the fact that great variability exists in terms of the chronologic age at which complete adult maturity is achieved in any given individual.

In an embodiment, at the time of detection or diagnosis of the cancer, the subject may be a premature infant, a newborn, aged between 0-12 months old, less than about 1 year old, less than about 2 years old, less than about 3 years old, less than about 4 less than about years old, less than about 5 years old, less than about 6 years old, less than about 7 years old, less than about 8 years old, less than about 9 years old, less than about 10 years old, less than about 11 years old, less than about 12 years old, less than about 13 years old, less than about 14 years old, less than about 15 years old, less than about 16 years old, less than about 17 years old, less than about 18 years old, less than about 19 years old, less than about 20 years old, less than about 21 years old, less than about 22 years old, less than about 23 years old or no more than about 24 years old. In an embodiment, the cancer is detected in the subject aged between 0-24 years old. In an embodiment, the cancer is detected in the subject aged between 0-21 years old. In another embodiment, the cancer is detected in the subject aged between 0-19 years old.

Where relevant in the description herein, the subject can have any type of cancer or tumor. In some embodiments, the cancer is a glioma, an Atypical Teratoid Rhabdoid Tumors (ATRT), an ependymoma (EPD), an Ewing sarcoma (ES), an Embryonal Tumor with Multilayered Rosettes (ETMR), a High-Grade Neuroepithelial Tumor (HGNET) or an osteosarcoma (OS). In another embodiment, the cancer is a synovial sarcoma, a rhabdomyosarcoma, a rhabdoid cancer, a medulloblastoma, a neuroblastoma, a hepatoblastoma, a paediatric germ cell cancer or a paediatric sarcoma. In some embodiments, the cancer is a medulloblastoma, a neuroblastoma, an astrocytoma, a pineoblastoma, a pleomorphic xanthoastrocytoma, a choroid plexus tumour, or a papillary tumour of the pineal region. In an embodiment, the cancer is a glioma. In an embodiment, the cancer is a high grade glioma. In an embodiment, the cancer is a paediatric high grade glioma. In another embodiment, the high grade glioma is a diffuse midline glioma or a diffuse hemispheric glioma. Other non-limiting examples of cancer include nasopharyngeal cancer, or cancer of the nasal cavity, oropharyngeal cancer, or cancer of the oral cavity, adrenal cancer, anal cancer, basal cell carcinoma, bile duct cancer, bladder cancer, cancer of the blood, bone cancer, a brain tumor, breast cancer, bronchus cancer, cancer of the cardiovascular system, cervical cancer, colon cancer, colorectal cancer, cancer of the digestive system, cancer of the endocrine system, endometrial cancer, esophageal cancer, eye cancer, gallbladder cancer, a gastrointestinal tumor, hepatocellular carcinoma, kidney cancer, hematopoietic malignancy, laryngeal cancer, leukemia, liver cancer, lung cancer, lymphoma, melanoma, mesothelioma, cancer of the muscular system, Myelodysplastic Syndrome (MDS), myeloma, nasal cavity cancer, nasopharyngeal cancer, cancer of the nervous system, cancer of the lymphatic system, oral cancer, oropharyngeal cancer, osteosarcoma, ovarian cancer, pancreatic cancer, penile cancer, pituitary tumors, prostate cancer, rectal cancer, renal pelvis cancer, cancer of the reproductive system, cancer of the respiratory system, sarcoma, salivary gland cancer, skeletal system cancer, skin cancer, small intestine cancer, stomach cancer, testicular cancer, throat cancer, thymus cancer, thyroid cancer, a tumor, cancer of the urinary system, uterine cancer, vaginal cancer, or vulvar cancer. The term “lymphoma” can refer to any type of lymphoma including B-cell lymphoma (e.g., diffuse large B-cell lymphoma, follicular lymphoma, small lymphocytic lymphoma, mantle cell lymphoma, marginal zone B-cell lymphoma, Burkitt lymphoma, lymphoplasmacytic lymphoma, hairy cell leukemia, or primary central nervous system lymphoma) or a T-cell lymphoma (e.g., precursor T-lymphoblastic lymphoma, or peripheral T-cell lymphoma). The term “leukemia” can refer to any type of leukemia including acute leukemia or chronic leukemia. Types of leukemia include acute myeloid leukemia, chronic myeloid leukemia, acute lymphocytic leukemia, acute undifferentiated leukemia, or chronic lymphocytic leukemia.

Examples of cancers that may be treated by the methods disclosed herein include cancers that cause solid tumors as well as cancers that do not cause solid tumors. Furthermore, any of the cancers mentioned herein can be a primary cancer (e.g., a cancer that is named after the part of the body where it first started to grow) or a secondary or metastatic cancer (e.g., a cancer that has originated from another part of the body). In an embodiment, the cancer to be treated is a primary cancer that is detected or diagnosed at a paediatric stage or adolescent stage. In another embodiment, the cancer to be treated is a secondary or metastatic cancer of the cancer that is detected or diagnosed at a paediatric stage or adolescent stage. This would include, for example, a secondary cancer or a metastasis that was detected in a non-paediatric or non-adolescent cancer, if the secondary cancer or metastasis arose from a cancer (whether a primary cancer or secondary metastasis) that was detected or diagnosed at a paediatric stage or adolescent stage (i.e. the secondary cancer or a metastasis was derived from or is otherwise related to the cancer that was detected or diagnosed at a paediatric stage or adolescent stage).

For the methods of treating a cancer or a tumor in a subject; methods of stratifying a subject having a cancer to a particular treatment regimen; and methods of predicting sensitivity to MCL1 inhibition treatments in a subject with cancer, as disclosed herein, the cancer is detected or diagnosed at a paediatric stage or adolescent stage. That is, the cancer is detected in the subject during the paediatric stage or adolescent stage. It is to be understood that the cancer formed and/or developed in the subject during the paediatric stage or adolescent stage. For example, for the methods of the present disclosure, the cancer is detected in a subject while they are no more than 24 years old. For example, for the methods of the present disclosure, the cancer is detected in a subject while they are no more than 21 years old. For example, for the methods of the present disclosure, the cancer is detected in a subject while they no more than 19 years old.

It is to be noted that, while the cancer is detected or diagnosed at a paediatric stage or adolescent stage, the methods of the present disclosure do not preclude treatment of the subject beyond the paediatric or adolescent stage. That is, treatment may commence and/or continue after the paediatric stage or after the adolescent stage. Treatment may commence and/or continue after detection or diagnosis of the cancer in the subject at the paediatric or adolescent stage, wherein the cancer comprises a cell that exhibits DNA hypermethylation at the BCL2L1 gene locus in comparison to the level of DNA methylation at a BCL2L1 gene locus in a non-cancerous cell. For example, where the cancer may have been detected or diagnosed in a patient while they are no more than 24 years old, and wherein the cancer comprises a cell that exhibits DNA hypermethylation at the BCL2L1 gene locus in comparison to the level of DNA methylation at a BCL2L1 gene locus in a non-cancerous cell the method of the present disclosure encompasses embodiments where treatment of the subject commences and/or continues beyond the time the subject is aged 24 years old, and into adulthood. For example, where the cancer may have been detected or diagnosed in a patient while they are no more than 21 years old, and wherein the cancer comprises a cell that exhibits DNA hypermethylation at the BCL2L1 gene locus in comparison to the level of DNA methylation at a BCL2L1 gene locus in a non-cancerous cell the method of the present disclosure encompasses embodiments where treatment of the subject commences and/or continues beyond the time the subject is aged 21 years old, and into early adulthood. In another example, the cancer may have been detected or diagnosed in a patient while they are no more than 19 years old, and wherein the cancer comprises a cell that exhibits DNA hypermethylation at the BCL2L1 gene locus in comparison to the level of DNA methylation at a BCL2L1 gene locus in a non-cancerous cell, treatment may commence and/or continues beyond the time the subject is aged 19 years of age. That is, the methods of the present disclosure contemplate methods of treatment (and stratification to treatment regimens), regardless of when treatment commences and/or occurs, as long as the cancer having BCL21 hypermethylation is detected in the subject at a paediatric or adolescent stage.

### 5. MCL1 Inhibition

The methods of the present disclosure generally relate to treating a cancer in a subject or stratifying a subject having a cancer to a particular treatment regimen, wherein the cancer detected in the subject at a paediatric or adolescent stage comprises a cell that exhibits DNA hypermethylation at the BCL2L1 gene locus in comparison to the level of DNA methylation at a BCL2L1 gene locus in a non-cancerous cell, and wherein the treating of the cancer or the treatment regime comprises an agent that inhibits MCL1 activity.

The unbiased targeted CRISPR-Cas9 loss-of-function screen of aHGG and pHGGs (as described herein) identified a robust and understudied dependency of pHGG on apoptosis regulator MCL1.

MCL1 (also known as BCL2L3; Bcl-2-Like Protein 3; Induced Myeloid Leukemia Cell Differentiation Protein Mcl-1; Myeloid Cell Leukemia Sequence 1 (BCL2-Related); Myeloid Cell Leukemia 1; or Bcl-2-Related Protein EAT/Mcll) encodes an anti-apoptotic protein, an antiapoptotic member of BCL-2 family proteins. The term MCL1 and MCL-1 are used interchangeably herein. The antiapoptotic function of MCL-1 is essential to cell survival and homeostasis. MCL1 is known to disrupt BAK/BAX/caspase 3,7-dependent apoptosis, and pharmacological inhibition by AZD5991/563845 which inhibits the binding of BAK and BAX to MCL1, resulting in cancer cell death.

As used herein, an agent that inhibits MCL1 activity refers to an agent that can be used to partially or completely reduce, suppress, interfere or inhibit with MCL1 expression, function and/or signaling. It is to be understood that the term “inhibit” and variations thereof such as “inhibition” and “inhibiting” do not necessarily imply the complete inhibition of signaling or function by MCL1. Rather, the inhibition may be to an extent, and/or for a time, sufficient to produce the desired effect. Inhibition may be suppression, retardation, reduction or otherwise hindrance of signalling by MCL1. Such inhibition may be in magnitude and/or temporal in nature. In particular contexts, the terms “inhibit” and “suppress,” and variations thereof may be used interchangeably. Examples of MCL1 inhibitors, and their mechanism of action include those disclosed in Xiang et al. (2018) Onco Targets Ther. 11:7301-7314, Tantawy et al. (2023) Front Oncol. 13:1226289; Bolomsky et al; (2020) J. Hematology & Oncology 13: 173; Hird and Tron (2019) Pharmacology & Therapeutics 198:59-67, and WO2021096860, the entire contents of which are incorporated herein by reference.

For example, the agent that inhibits MCL1 activity may be a small molecule that binds MCL1 and prevents its interaction with other proteins. In particular, the agent that inhibits MCL1 activity may be a small molecule that binds MCL1 and disrupts its interaction with BAK. In another example, the agent that inhibits MCL1 activity may be a small molecule that binds MCL1 and prevents normal MCL1 signaling. In another example, the agent that inhibits MCL1 activity may be an antibody or an antigen binding fragment thereof, that binds to MCL1 and prevents normal MCL1 function/activity.

MCL-1 is a short-lived protein, and its expression is tightly regulated at transcriptional, translational, and post-translational levels (Nijhawan et al. (2003) Gene Dev. 17:1475). In some examples, the agent that inhibits MCL1 activity can be an agent that targets the inactivation of MCL1 genetic sequence (e.g. targeted gene editing) or inactivation of MCL1 transcripts (e.g. siRNAs, microRNAs, antisense oligonucleotides). In some examples, the agent that inhibits MCL1 activity may be an agent directly or indirectly targets MCL-1 expression to downregulate MCL1 expression. For example, inhibition of MCL-1 upstream signal pathways can downregulate MCL-1 expression by decreasing transcription and translation of MCL1. For example, CDK9 inhibitors, roscovitine and CR8, and Na+/K+-ATPase inhibitor cardiac glycosides UNBS1450 effectively inhibit MCL-1 transcription. Benzyl isothiocyanate inhibits the phosphorylation of eukaryotic initiation factor 4G, resulting in a decreased MCL-1 translation, followed by cell cycle arrest and apoptosis in leukemia cells. Other MCL1 translation inhibitors include PI3K/mTOR inhibitors (e.g. BEZ235 and AZD8055) and EGFR/VEGFR inhibitors (BAY43-9006). Promotion of MCL-1 degradation is another approach to reduce cellular MCL-1 protein levels; e.g. activation of GSK-33 phosphorylation by arsenic trioxide and bufalin enhances MCL-1 degradation. Other agents known to promote degradation of MCL1 protein include ERK inhibitors and WP1130.

Illustrative examples of other MCL-1 inhibitors include 7-(5-((4-(4-(N,N-Dimethylsulfamoyl)piperazin-1-yl)phenoxy)methyl)-1,3-dimethyl-1H-pyrazol-4-yl)-1-(2-morpholinoethyl)-3-(3-(naphthalen-1-yloxy)propyl)-1H-indole-2-carboxylic Acid, S63845, omacataxine, seliciclib, UMI-77, AT101, sabutoclax, TW-37, MIK665/564315, AMG-176, VU661013, AMG-397, ABBV-475, ABBV-467, ANJ810, TTX-180, ABBV-467 and/or PRT1419, marinopyrrole A, A-1210477, Sabutoclax (BI-97C1). Thus, in an embodiment, the agent that inhibits MCL-1 is selected from the group consisting of 7-(5-((4-(4-(N,N-Dimethylsulfamoyl)piperazin-1-yl)phenoxy)methyl)-1,3-dimethyl-1H-pyrazol-4-yl)-1-(2-morpholinoethyl)-3-(3-(naphthalen-1-yloxy)propyl)-1H-indole-2-carboxylic Acid, S63845, omacataxine, seliciclib, UMI-77, AT101, sabutoclax, TW-37, MIK665/564315, AMG-176, VU661013, AMG-397, ABBV-475, ABBV-467, ANJ810, TTX-180, ABBV-467 and/or PRT1419, marinopyrrole A, A-1210477, Sabutoclax (BI-97C1), and a combination of one or more of the foregoing.

In an embodiment, the agent that inhibits MCL-1 activity is an MCL1-siRNA, an MCL-1 antisense oligonucleotide, MCL1 targeted gRNAs, S64315, S63845, AZD5991, AMG-176, AMG-397, ABBV-475, ANJ810, TTX-180, ABBV-467 and/or PRT1419.

In an embodiment, the agent that inhibits MCL1 activity is MCL-1 targeted gRNAs. In an embodiment, the MCL1 targeted gRNAs have the nucleotide sequence of SEQ ID NO: 2 and SEQ ID NO: 2.

In an embodiment, the agent that inhibits MCL1 activity is S63845. In an embodiment, the agent that inhibits MCL1 activity is AZD5991.

It is also contemplated that for the methods of treatment of the present disclosure, the agent that inhibits MCL1 may be administered as a single agent. Alternatively, the agent that inhibits MCL1 may be administered as a single agent or may be administered in combination with, or in a treatment regimen comprising, one or more of anti-cancer drugs, chemotherapy, radiotherapy, surgery, adjuvant therapy, and neoadjuvant therapy drugs. For example, the treatment may comprise an agent that inhibits MCL1, and one or more of BH3 mimetics (e.g. BCL inhibitors such as ABT-737, ABT-199, ABT-263), epigenetic modifying agents (e.g. azacytidine, decitabine, Temozolamide) topoisomerase inhibitors such as etoposide, cyclin-dependent kinase inhibitors, kinesin-spindle protein stabilizing agents, proteasome inhibitors, tyrosine kinase inhibitors, modulators of cell cycle regulation (by way of non-limiting example, a cyclin dependent kinase inhibitor), modulators of cellular epigenetic mechanistic (by way of non-limiting example, histone deacetylases (HDAC) (e.g. one or more of vorinostat or entinostat), an anthracycline or anthracenedione (by way of non-limiting example, one or more of epirubicin, doxorubicin, mitoxantrone, daunorubicin, idarubicin), platinum-based therapeutics (by way of non-limiting example, one or more of carboplatin, cisplatin, and oxaliplatin), cytarabine or a cytarabine-based chemotherapy. In an embodiment, the MCL1 inhibitor is administered in combination with, or in a treatment regimen comprising Temozolamide, as described, for example, in Gratas et al. (2014, Oncotarget 5:2428-2435), the entire contents of which is incorporated herein by reference.

It will be appreciated by persons skilled in the art that numerous variations and/or modifications may be made to the present disclosure without departing from the spirit or scope of the disclosure as broadly described. The present embodiments are, therefore, to be considered in all respects as illustrative and not restrictive. The invention also includes all of the steps, features, compositions and compounds referred to or indicated in this specification, individually or collectively, and any and all combinations of any two or more of said steps or features.

The present disclosure will now be further described in greater detail by reference to the following specific examples, which should not be construed as in any way limiting the scope of the disclosure.

### EXAMPLES

### Materials and Methods

### Cell Lines and Culture Conditions

Suspension cell lines were collected and centrifuged at 1200 RPM for 5 minutes to remove supernatant. Accutase (Thermo Fisher, Gibco, Life Technologies) was added to cell pellet, mixed, and incubated at 37° C. 5% CO2 for ˜5 minutes. Media was added to inactivate Accutase, centrifuged to remove supernatant and then resuspended in fresh media. Adherent cell lines were washed with PBS then incubated with TrypLE Select Enzyme (1X) (Thermo Fisher, Life Technologies) at 37° C. 5% CO2 for ˜5 minutes to allow cell detachment. Fresh media was then added to inactivate the TrypLE Select Enzyme before centrifugation and resuspension in fresh media. All cells were cultured aseptically at 37° C. in humidified incubators with 5% CO₂.

### Lentiviral Production

Lentivirus production for MCL1 sgRNA, BCL2L1 sgRNA and BCL2L1 overexpression vector were as follows: On day 1 HEK293T cells were seeded and incubated overnight at 37° C. The following day, a mixture of 1.2 g psPAX (Addgene; #12260), 0.6 g pMD2 (Addgene; #12259) and 1.2 g of vector plasmid was diluted in 500 μL of Opti-MEM (Gibco) and 10 L Lipofectamine 2000 (Invitrogen) and incubated at room temperature for 30 minutes. The transfection solution was then added dropwise to HEK293T cells and incubated overnight at 37° C. The following day, media was replaced with 30% FBS DMEM media and incubated for a further twenty-four hours where viral containing supernatant was collected at 24- and 48-hour time points. The virus was further concentrated with Lenti-X concentrator (Takara Bio), resuspended in PBS then aliquoted and stored at −80° C.

### Virus Transduction

Lentivirus was added to cells with polybrene (2 μg/mL) (Thermo Fisher, Invitrogen, Life Technologies, Netherlands, Europe) before incubation overnight at 37° C. Twenty-four hours following transduction, selection was initiated with either blasticidin (5 μg/mL) or puromycin (2 g/mL) (Thermo Fisher, Invitrogen, Life Technologies, Netherlands, Europe).

Establishment of modified cell lines

Two MCL1 or BCL2L1 targeted sgRNAs were cloned in to a pLenti guide-puro vector (Addgene #52963) using the Golden Gate cloning method, and lentivirus was produced for each guide as described previously (23). AURKB sgRN A (cell-killing positive control) and Non-targeting control sgRNA (negative control) were previously cloned and sequence validated. For validation experiments, Cas9 2A-Blast cells were seeded for each condition then transduced 24 hrs later. Puromycin (2 ug/mL) selection was maintained from day 3 to day 7 when viability was assessed using an alamarBlue assay. BCL2L1_PLX307 (BCL2L1 overexpression vector) corresponds to Addgene plasmid #98323. sgRNA sequences (5′ to 3′); MCL1 sgRNA #1-AGGCGCTGGAGACCTTACGA (SEQ ID NO: 1), MCL1 sgRNA #2-GTAATAACACCAGTACGGAC (SEQ ID NO: 2), BCL2L1 sgRNA #1-CAGGCGACGAGTTTGAACTG (SEQ ID NO: 3), BCL2L1 sgRNA #2-CTCCGATTCAGTCCCTTCTG (SEQ ID NO: 4).

### Western Blotting

Cells were harvested and lysed with RIPA buffer. After measuring the protein concentration using the Bicinchoninic acid assay (BCA assay) (Thermo Fisher, Life Technologies, Netherlands, Europe), the required amount of protein was separated using 4-12% sodium dodecyl sulphate-polyacrylamide (SDS-PAGE) gel (Bolt™, Life Technologies, Netherlands) then transferred to the Polyvinylidene difluoride (PVDF) membrane. The membrane was blocked with blocking buffer (LI-COR, Biosciences, US) then incubated with primary antibody at 4° C. overnight, followed by incubation with a secondary antibody for 1 hour before visualization (Odyssey; LI-COR, Biosciences, US). 3-actin was used as a loading control. Primary antibodies: MCL1 (#32087, Abcam), BCL2L1 (#2764, CST), j-Actin (Bio-Rad). Secondary antibodies: Goat anti-rabbit 680 (LI-COR) and Goat anti-rabbit 800 (LI-COR).

### Targeted Pooled CRISPR-Cas9 Loss of Function Screens

Targeted pooled CRISPR-Cas9 screens performed on cell lines in this study were executed according to a previous study (22). In vitro screens; In brief, customized sgRNA library consisting of 1666 sgRNAs corresponding to 352 target genes at 4 sgRNAs per gene was utilized to perform pooled knockout genetic screens. The genes included 168 oncology drug targets, 66 oncology preclinical targets, 56 cancer genes, 62 core essential genes (positive controls) and two non-targeting negative control genes with 250 sgRNAs. To achieve a 500x sgRNA representation (lx sgRNA to infect 500 cells), sgRNAs were introduced into Cas9-expressing cell lines in replicate via lentiviral transduction at a multiplicity of infection (MOI) of 0.3. Positively transduced cells were selected using 2 g/mL puromycin throughout the 21-day screen. In vivo screens; 2.5 million cells infected with the previously described sgRNA library were transplanted intracranially in NOD SCID Gamma (NSG) immunodeficient mice. The mice were observed daily for 7 days, then thrice weekly for signs of neurological distress or 20% weight loss. At ethical endpoints, mice were euthanized, and their brains were harvested. Following the collection of cells and tumor samples, genomic DNA was obtained using the Qiagen DNeasy Blood & Tissue kit. In each sample, the sgRNA library was amplified using a P5 forward and a uniquely barcoded P7 reverse primer (IDT), resulting in a −360 bp amplicon confirmed by agarose gel electrophoresis. Finally, PCR products for each sample were pooled and purified using AMpure Beads, and the amplicons along with the sgRNA library plasmid controls were submitted for next-generation sequencing.

### Caspase Glo 3/7 Assay

Following drug treatment, cells were subjected to caspase 3/7 activity measurement using the Caspase-Glo 3/7 assay kit (Promega, Madison, USA). White-walled 96-well plates containing cells were removed from the incubator and allowed to equilibrate to room temperature for 30 minutes. Then, 50 ul of Caspase-Glo reagent was added to each well, and the contents of the wells was gently mixed by a plate shaker at 300-500 rpm for 30 seconds. After 2 hours of incubation in room temperature, the luminescence of samples was measured with the Clario Star plate reader (BMG, LABTECH). Readings were normalized to cell-viability alamarBlue assay. The experiments were performed in triplicate and repeated with three separately initiated cultures.

### DNA Extraction and Bisulphite Treatment

DNA was extracted from HGG cell lines and patient tissue samples using the DNeasy Tissue Kit (Qiagen Ltd) and subsequently bisulphite-treated using the EZ DNA Methylation-Kit (Zymo Research) according to the manufacturer's protocol to convert all unmethylated cytosine to uracil while leaving 5-methylcytosine unaltered. The treated DNA was then eluted in 12 μL of M-elution buffer.

### Pyromark-PCR Assay and Pyrosequencing

PCR and sequencing primers were designed using the PyroMark Assay Design Software.

PCR assays were designed to amplify the CpG site of interest (BCL2L1: cg0020098) for subsequent pyrosequencing. Hot-Star PCR was carried out with the HotStar Taq Master Mix Kit (Qiagen Ltd.) using 500 ng of bisulphite-treated DNA along with a negative control. Confirmation of PCR product quality was established on a 2% agarose gel with ethidium bromide staining. Pyrosequencing was performed using the PyroMark Q48 Autoprep instrument (Qiagen Ltd). PCR and sequencing primer sequences (5′ to 3′); Forward primer-TTTTATTTGTTTTTTTTAAGGGGTTTTAGT (SEQ ID NO: 5), Reverse-Biotin primer-TCCTACCTATAACCATACCCTAATCT (SEQ ID NO: 6), Sequencing primer-AAGTTTTTTTTATTTTAAAGTTTG (SEQ ID NO: 7).

### Drug Sensitivity Assay

AZD5991 (#S8643) and S63845 (#C-1370) from Selleckchem (Selleckchem, TX, USA) and Active Biochem (Assay Matrix, VIC, AUS) were dissolved in dimethyl sulfoxide (DMSO) and stored at −80° C. where they were freeze-thawed up to five times prior to disposal. Cells were seeded in triplicate into 96-well plates then treated at concentrations ranging from 0-20 μM. Cell viability was assessed after 72 hours by an alarmBlue assay.

### Immunohistochemistry (IHC) and Imaging Analysis

Immunohistochemical staining for MCL1 was performed by the Monash Histology Platform, Monash University using the avidin-biotin-peroxidase complex (ABC) method. Adult and paediatric HGG TMAs obtained from US Biomax Inc. were deparaffinized for IHC. Briefly, following deparaffinization and antigen retrieval by microwaving in ethylenediaminetetraacetic acid (EDTA) buffer for 20 minutes, endogenous peroxidase activity was blocked by 0.3% H₂O₂ in methyl alcohol for 30 minutes. Subsequently, primary antibody MCL1 (Abcam #32087) was applied at a dilution of 1:2000 overnight at 4° C. The sections were then incubated with a biotinylated secondary antibody diluted 1:300 in PBS for 40 minutes followed by washing with PBS. Next, the color reaction was carried out with DAB and nuclei were counterstained with hematoxylin.

The scanned images were analyzed by Aperio ImageScope 12.3.3 software. The positivity threshold for staining was determined empirically based on controls and the intensity was classified from 0 to 3.

### Predictive Modelling of Drug Response Using Random Forest Machine Learning Algorithm

To predict biomarkers for AZD5991 sensitivity, the machine-learning Random Forest (RF) method was used to build predictive models based on multi-omics datasets (https://github.com/broadinstitute/cdsr_models). In brief, for AZD5991 drug response, the top 400 correlated features were filtered to fit into a predictive model. The features included transcriptomic profiles (top 8000 variable genes), cancer-related damaging mutations and copy number variations (CNV), methylation (top 8000 variable CpG sites) and clinical information (cancer types, gender, and age-group). HGG cell lines were classified into either sensitive or resistant groups using the threshold drug sensitivity value of LogIC50 3.5/IC50 2 μM, and the AUC was calculated using Graph Pad Prism 9.4.1.

RF relative importance was obtained by calculating the gradient of the line of best fit between ranked importance values for each random forest model. The features with the greatest contribution were obtained by dividing the gradient by the maximum importance value and the total length of features assigned to the model. Features with a derived score of >=0.3 were considered a top contributing feature to the random forest and used to normalize importance.

### Mouse Strains

All animal experiments utilized 6 to 10-week-old NOD.Cg-Prkdc^{scid}IL2rg^{tmlwjl}/SzJ (NSG) mice. Mouse colonies were bred and maintained in-house at the Hudson Institute of Medical Research Animal Facility (Clayton, Victoria, Australia) under specific-pathogen-free (SPF) conditions. All animal experiments and research plans were approved by the Animal Ethics Committee (MMCA/2022/13—Modelling Paediatric Brain Tumors in vivo).

### Quantification and Statistical Analysis

All statistical analysis was performed using GraphPad Prism software V9.0. or R (R version 4.2.0) with appropriate tools/packages. IC50s of drug response were estimated using GraphPad Prism. The significant difference between two groups was analyzed using the unpaired Student's t-test, and for multiple comparisons, one-way ANOVA with Turkey post-test/Dunnett-test was conducted. p<0.05 was considered statistically significant. Statistical tests were adjusted for multiple hypotheses correction using the Benjamini-Hochberg False Discovery Rate (FDR) with <5% considered significant. Correlation between variables was calculated based on the Pearson rank correlation coefficient. Quantified multi-omics datasets (CRISPR dependencies, transcriptomics and DNA methylation) were obtained from (22). Genetic dependencies were determined by comparing sgRNA representation (β-score) on day 21 to day 0 plasmid reference. Genes with mean β-score<=0.5 were defined as ‘hits’, signifying reduced cell viability. Unpaired t-tests analyzed β-score across 352 genes in paediatric and adult HGG cell lines, revealing unique vulnerabilities. Hits specific to pHGG or aHGG were defined using Δ mean β-score±0.1, FDR<0.05. Relevant statistical parameters are stated in the legend of each figure.

### Example 1: MCL1 is an Enriched Gene Dependency in Paediatric High-Grade Gliomas

A pooled CRISPR-Cas9 loss-of-function screen was performed across a large cohort of adult (n_{(aHGG)}=10) and paediatric (n_{(pHGG)}=65) HGG cell lines.

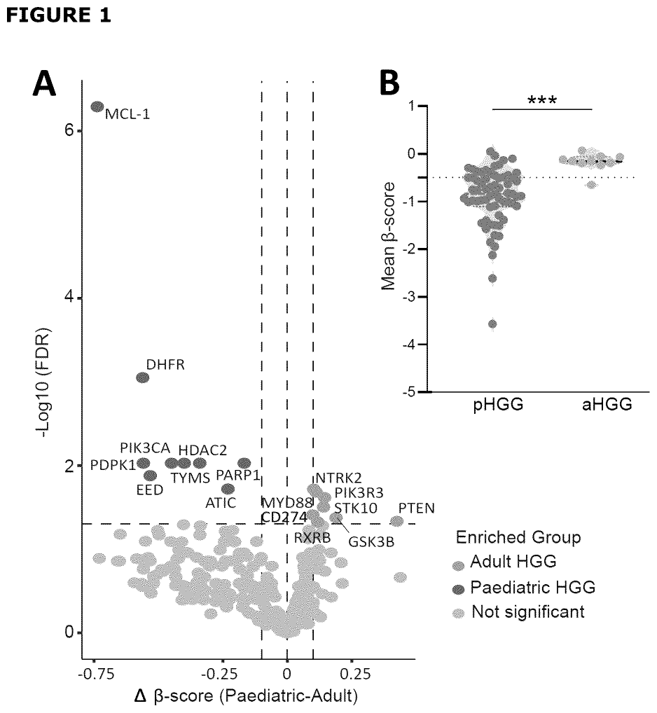

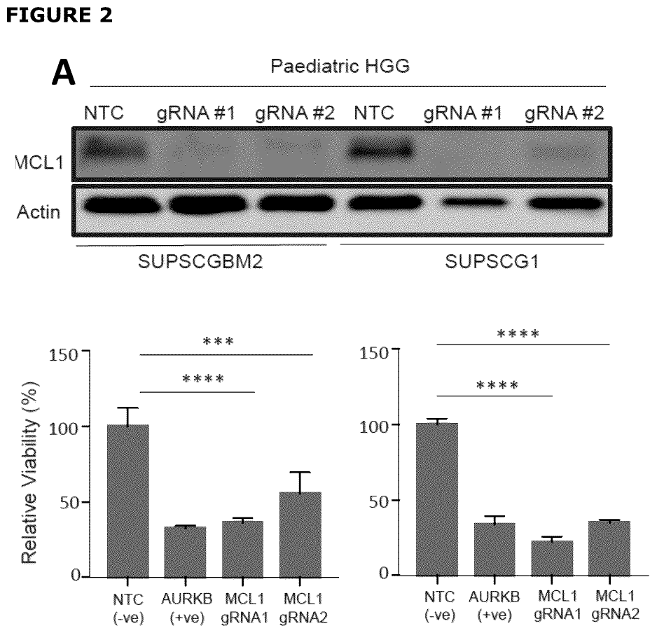

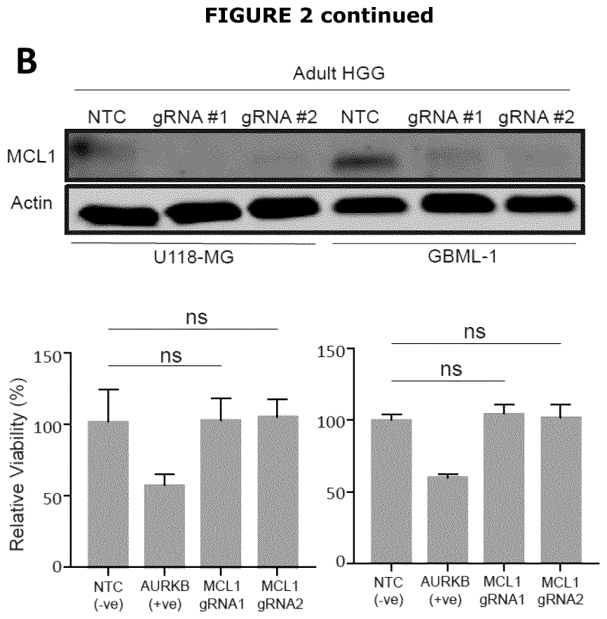

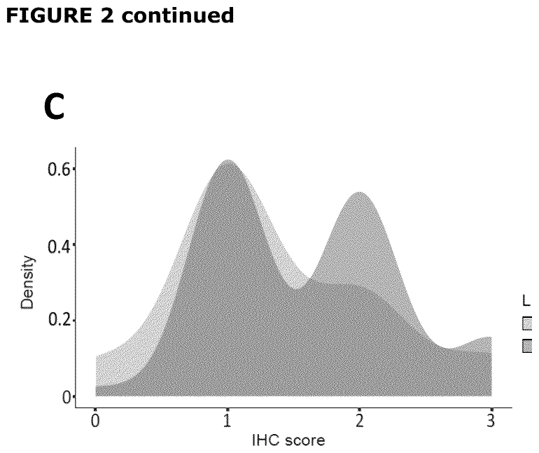

Seventeen genes were found to have age-specific growth effects, eight of which were specific for aHGGs (MYD88, PIK3R3, NTRK2, CD274, STK10, PTEN, GSK3B and RXRB) and nine which were distinctly required for cellular fitness in pHGGs (MCL1, DHFR, PDPK1, EED, PIK3CA, HDAC2, ATIC, PARP1, and TYMS) (FIG. 1A). Of these, MCL1 was the single most enriched growth dependency in pHGGs (aHGG=−0.18, pHGG=−0.92, p=0.0005). Comparison of gene dependency across the HGG cohort revealed that MCL1 dependency was significantly more prevalent in pHGG (75%; 49 of 65) compared to aHGG cell lines (10%; 1 of 10) (FIG. 1B). Consistent with the screen data, knockout (KO) of MCL1 by two independent sgRNAs led to the loss of viability in pHGG cell lines, SUPSCGBM2 and SUPSCG1 (FIG. 2A; p<0.05). AURKB was used as a positive control. In contrast, MCL1 depletion had no growth effect in either aHGG lines, U118-MG, and GBML-1 (FIG. 2B; p=ns), confirming MCL1 as an age-related dependency in HGG.

To explore whether there are expression disparities related to MCL1 dependency in HGGs, MCL1 expression levels was expressed by immunohistochemistry (IHC) on a tissue microarray of adult (n=71) and paediatric (n=172) HGG tumors which were scored for MCL1 expression. Immunohistochemical staining of MCL1 was categorized as absent (IHC score 0), low (IHC score 1), moderate (IHC score 2), and high (IHC score 3) (see Table 1 below).

TABLE 1
IHC analysis of adult and paediatric HGG tumors

| | MCL1 Expression | | |
|---|---|---|---|
| | Low expression | High expression | |
| | (IHC score 0 & 1) | (IHC score 2 & 3) | Total |
| Adult HGG | 46 | 25 | 71 |
| Paediatric HGG | 83 | 89 | 172 |
| Total | 129 | 114 | 243 |

The results show that 52% (89/172) of pHGG tumor cores exhibited moderate-high MCL1 staining (score≥2) compared to adult tumors where only 35% (25/71) of the cores expressed moderate-high levels of MCL1 (FIG. 2C and Table 1; p=0.02). The expression data suggested that MCL1 is a paediatric glioma enriched genetic dependency.

### Example 2: MCL1 Inhibitors AZD5991/S63845 Selectively Kill Paediatric HGG Cell Lines In Vitro

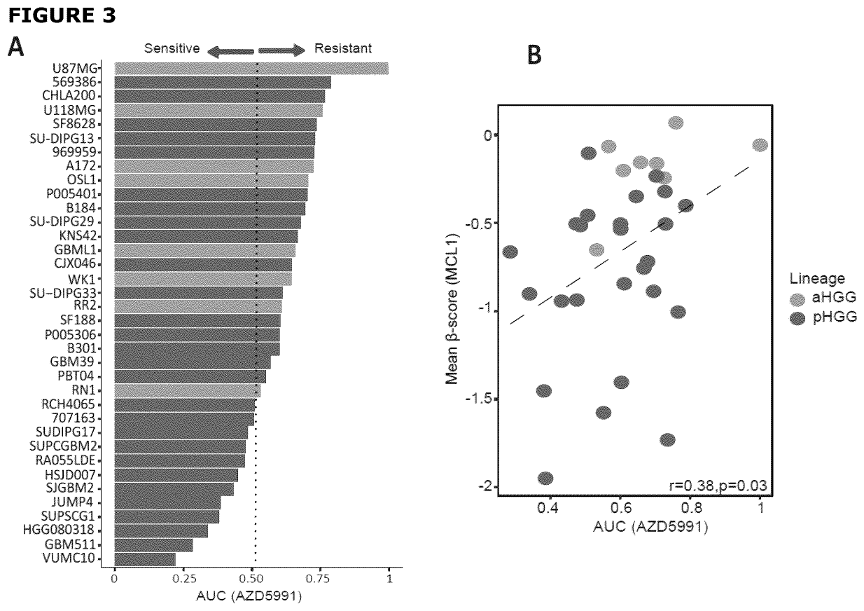

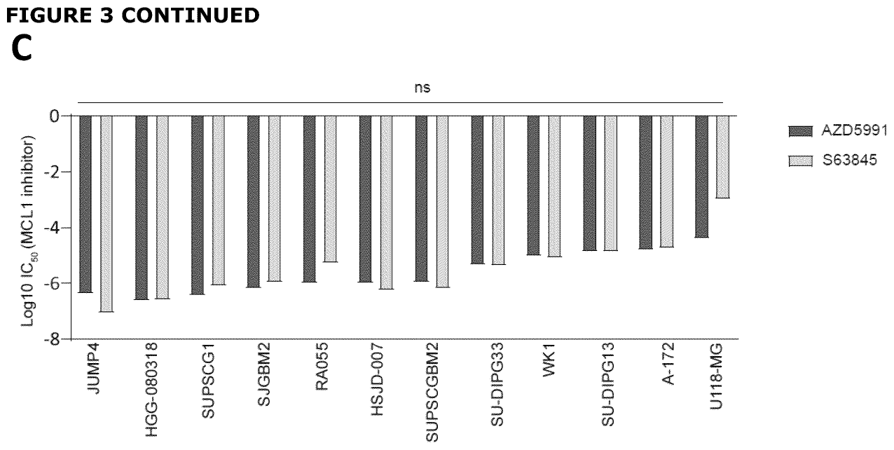

A panel of 36 paediatric and adult HGG cell lines was subjected to 12-point dose treatments (0-20 μM) using the MCL1 inhibitor AZD5991. Consistent with the CRISPR screening data, MCL1 inhibition was broadly more effective (IC50<2 PM, AUC<0.5) across paediatric (n=12/28;43%) versus adult (n=0) HGG lines (FIG. 3A). A proportion of paediatric cell lines (n=7/28;26%) demonstrated exquisite sensitivity to AZD5991, with antineoplastic effect observed at low nanomolar concentrations. In contrast, response to AZD5991 was observed only at concentrations greater than 2 μM in aHGGs (FIG. 3A; AUC>0.5).

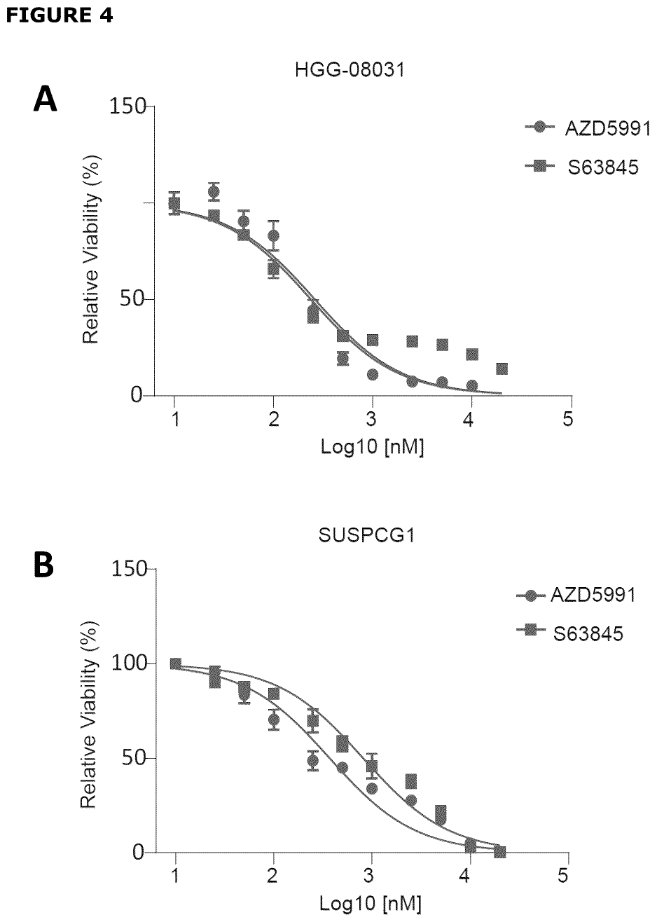

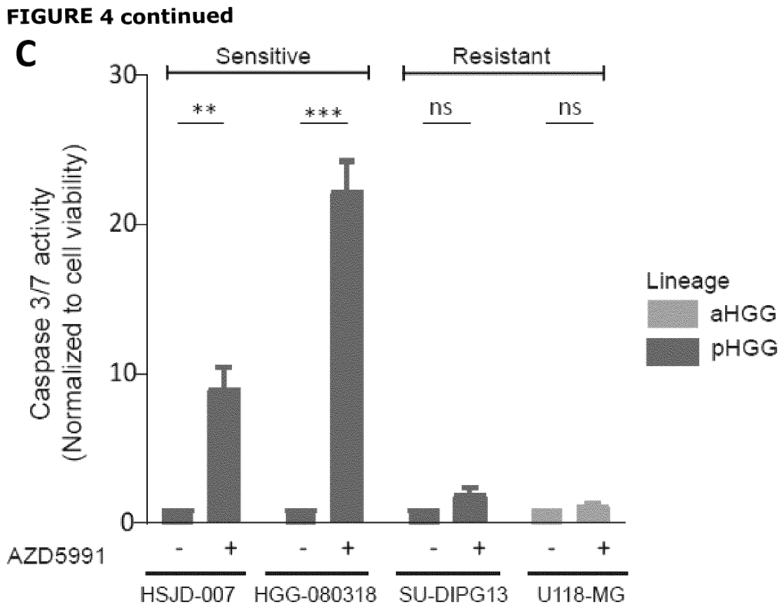

Comparing MCL1 gene dependency scores with AZD5991 drug effect revealed a modest, yet statistically significant correlation (FIG. 3B; r-0.38, p=0.03). MCL1 inhibitory effects in HGG cells were investigated using an independent MCL1 inhibitor, S63845 which showed a similar activity to AZD5991 in HGG lines (FIG. 3C, FIG. 4A-B). Assessment of caspase-mediated cell death confirmed that MCL1 inhibition led to increased caspase 3/7 cleavage and apoptosis (p<0.001), an effect not observed in cell lines resistant to AZD5991 (p=ns) (FIG. 4C). These findings collectively indicate that MCL1 inhibitors exhibit potent effects on pHGG.

### Example 3: a Novel Cluster of CpG Methylation Sites Predicts MCL1 Inhibitor Response in Paediatric High Grade-Gliomas

Unbiased Artificial Intelligence (AI)-based approach was used to identify biomarkers associated with MCL1 inhibitor response in pHGGs. Random Forest (RF) machine learning models (Dempster et al. 2020 BioRxiv 2020.02. 21.959627) were used to predict AZD5991 drug response in pHGG using RNA expression (top 8000 variable genes), DNA methylation (top 8000 variable CpG sites), DNA point mutations, copy number variations (CNVs) and clinical information (cancer types, gender, and age-group) as input features. Model performance was evaluated by comparing the predicted drug response with the actual response in pHGG cell lines. Features were ranked according to their computed importance score, reflecting each feature's contribution to the predictive performance of the model.

Remarkably, among the 140 predictive features identified, one of the top features associated with MCL1 inhibitor (AZD5991) response in pHGGs was a previously undescribed methylation site, cg00300298 within gene BCL2L1. Single-correlate analysis conducted between the predictive features and MCL1 inhibitor activity (AUC of AZD5991) found that cg00300298 was ranked as the 4th most highly associated feature of AZD5991 response in pHGGs.

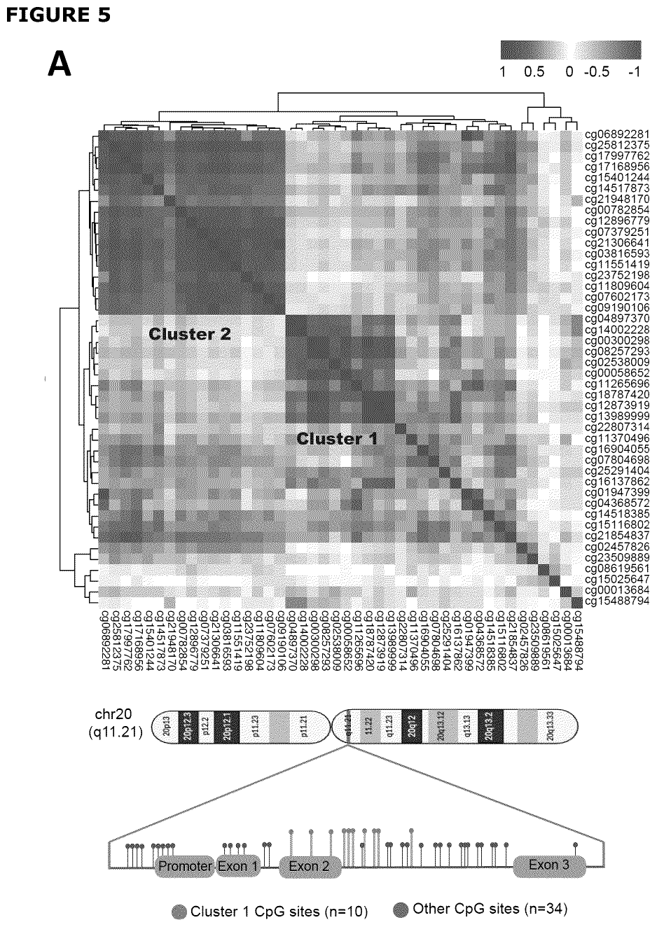

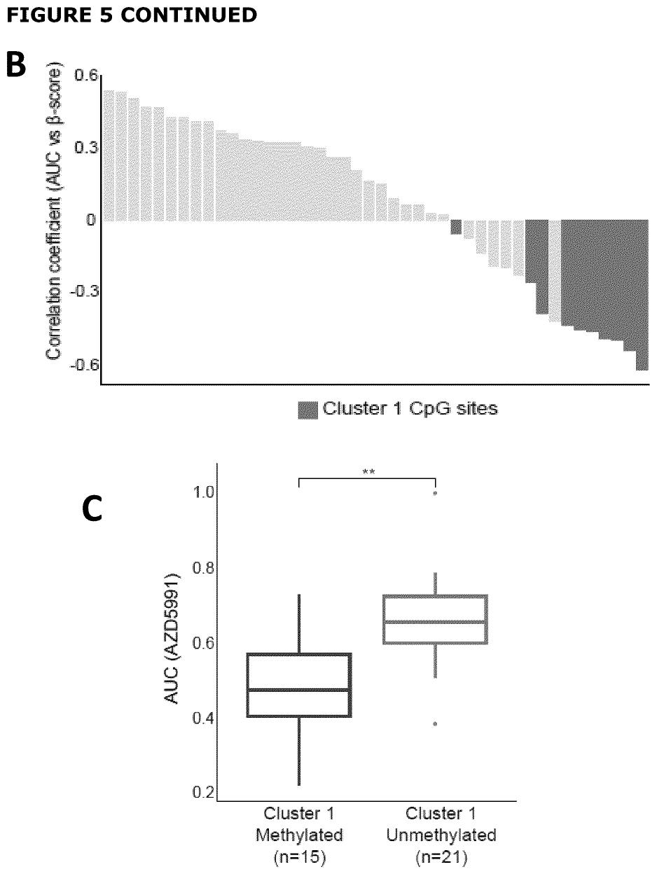

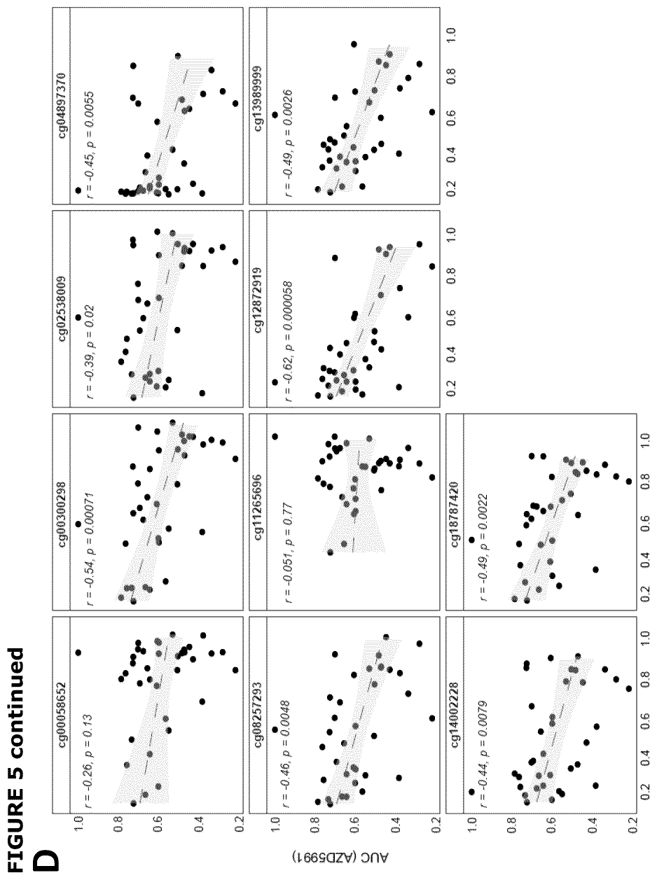

The methylation status of 44 CpG sites mapping to the BCL2L1 locus in the childhood cancer model atlas cohort of 238 cell lines were examined, using the Infinium methylation array. Hierarchical clustering of the 44 BCL2L1-associated methylation sites, identified a cluster of 10 CpG sites (cluster 1), including cg00300298, that were tightly correlated. (FIG. 5A). An additional cluster of sites (cluster 2) corresponded to sites that were uniformly lowly. Interestingly Cluster 1 sites mapped at the exon 2 and intron 2 junction of the BCL2L1 locus and upstream of the long non-coding RNA apoptotic BCL2L1-antisense long non-coding RNA (ABALON), a known regulator of BCL2L1 isoform expression. To validate these findings, single-correlate analysis was conducted between the methylation scores of the 44 BCL2L1 CpG sites and MCL1 inhibitor activity (AUC of AZD5991) across the cohort of pHGGs.

Cluster 1 CpG sites mapped as the most highly correlated features. Notably, cg00300298 ranked 2nd with a significant correlation coefficient (r) score −0.54 and p=0.00071 (FIG. 5B, FIG. 5D). Consistently, defining the cell line cohort as methylated versus unmethylated (beta-score cutoff>0.5), a significant association between Cluster 1 methylation and MCL1 inhibitor activity (FIG. 5C; p=0.0013) was observed. These results identify methylation within the BCL2L1 gene locus (cluster 1 CpG sites) as a predictive marker for MCL1 inhibitor response in pHGGs.

### Example 3: Cg00300298 DNA Methylation in Paediatric CNS Tumors

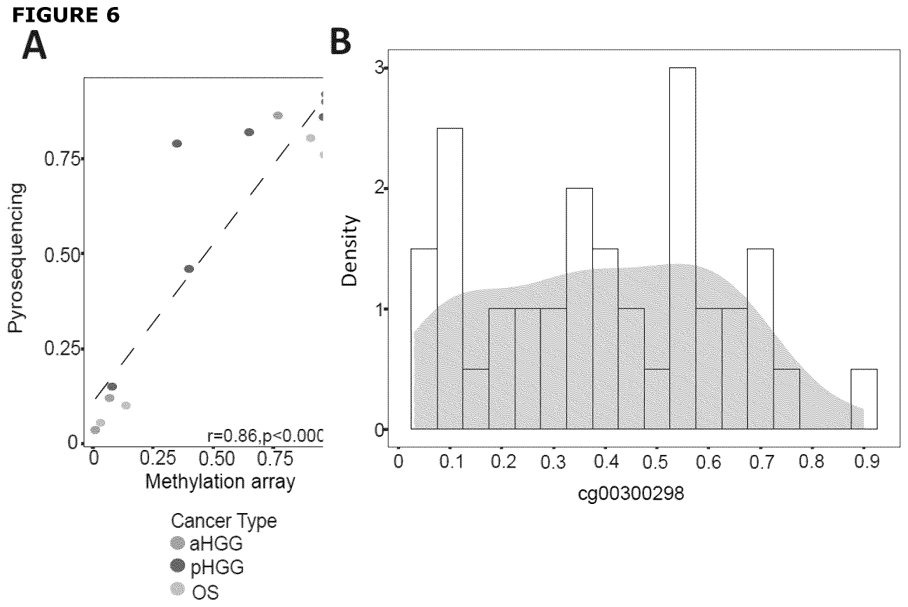

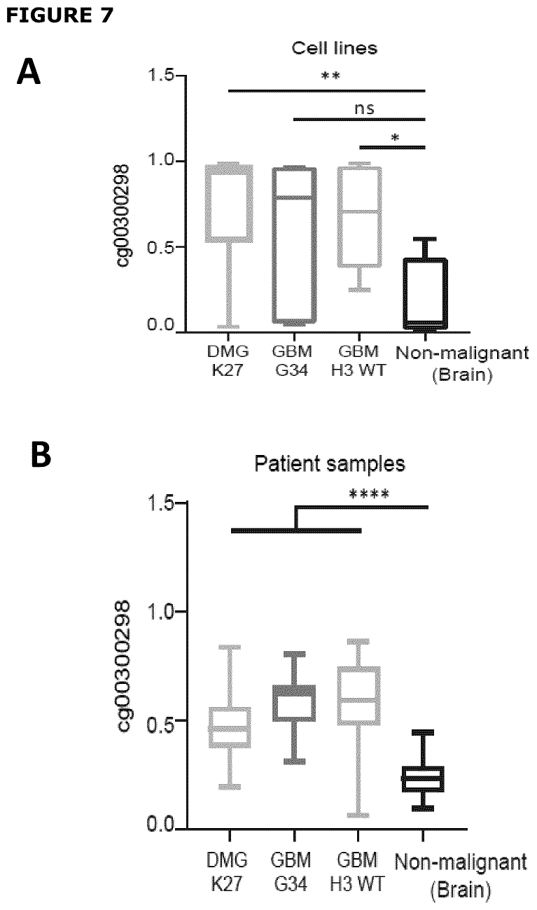

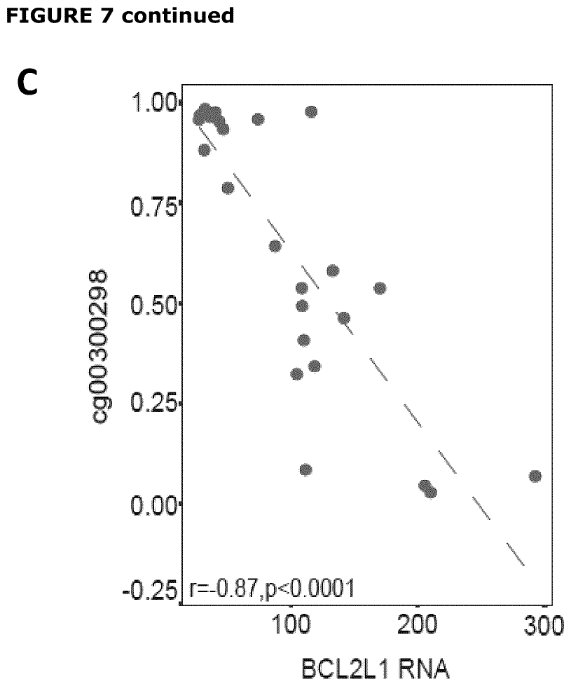

Utilizing cg00300298 as an exemplar CpG site within the cluster, its methylation status was validated through pyrosequencing across the spectrum of HGG cell lines. Pyrosequencing derived methylation scores strongly correlated with Infinium methylation array readings and confirmed the predictive value of cg0030098 methylation state for MCL1 inhibitors (FIG. 6A; r=0.86; p<0.0001). Methylation was subsequently analyzed across a cohort of pHGG (n=72) patient tissue samples from which 40 (55%) passed quality control. An even distribution of methylation at cg00300298 was observed across the cohort of pHGG samples (FIG. 6B). Comparison of pHGG to non-malignant tissue specimens showed cg00300298 hypermethylation, confirming that BCL2L1 was hypermethylated in both pHGG cell lines (FIG. 7A; p=0.005) and tumor samples (FIG. 7B; p<0.0001). Interestingly, cg00300298 methylation was similar across all 3-histone altered pHGG subtypes, indicating that MCL1 dependency is a common phenotype across a spectrum of childhood gliomas.

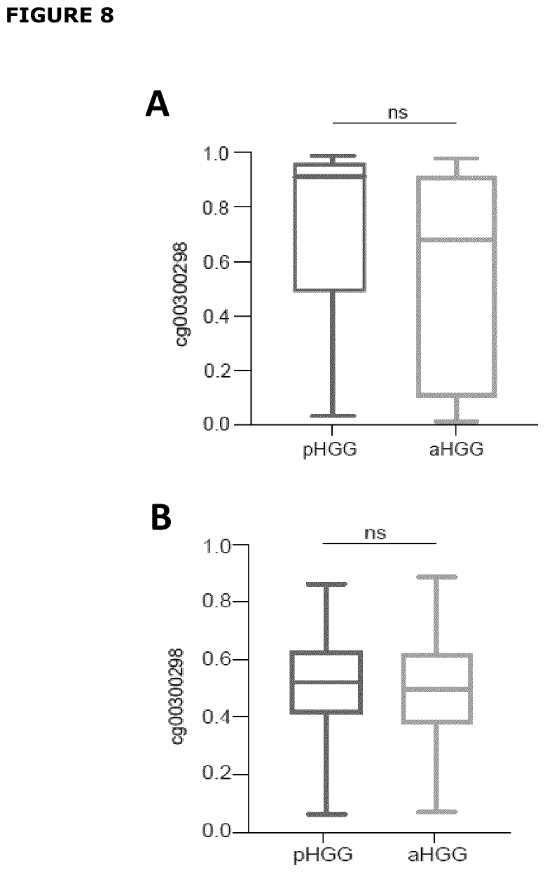

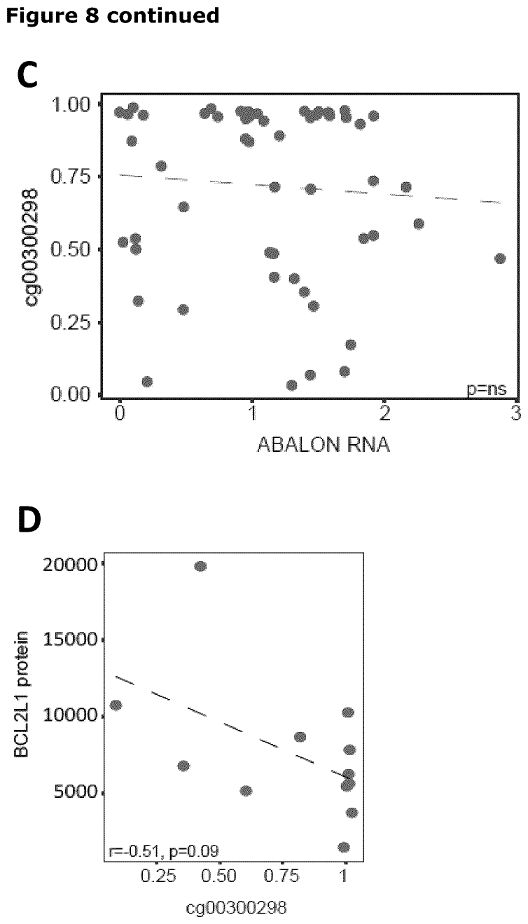

The analysis was expanded to aHGG to investigate whether methylation differences underline the observed ubiquitous lack of dependency on MCL1. Both adult and paediatric HGGs showed similar methylation status at cg00300298 in cell lines (FIG. 8A; p=ns). This was broadly recapitulated in patient tumors where adult gliomas harbored similar methylation at the cg00300298 site compared to childhood gliomas (FIG. 8B; p=ns). Deletion of MCL1 in hypermethylated adult tumors (n=5; 50%) was ineffective, consistent with the inability of cg00300298 methylation to inform MCL1 dependency in adult HGGs.

The associations between cg00300298 methylation status and expression from proximally located genes was explored to derive insight into how it may functionally contribute to determining MCL1 dependency. Two loci of note were identified which could feasibly represent cis-regulated gene products of cg00300298: BCL2L1 and ABALON. ABALON is a long non-coding RNA which regulates splicing of BCL2L1 pre-mRNA to induce preferential expression of the pro-apoptotic protein product BCL2-xs isoform over the anti-apoptotic gene product BCL2-XL. The cg00300298 locus is −354 bp upstream of the ABALON transcription start site. A poor correlation was observed between cg00300298 methylation and ABALON mRNA expression (FIG. 8C, p=ns). The expression of BCL2L1 itself in the context of cg00300298 methylation was explored. There was a strong inverse correlation between cg00300298 methylation and BCL2L1 RNA levels (FIG. 7C; r−0.87, p<0.0001). Interestingly, when examining protein levels, this relationship between cg00300298 methylation and protein expression was lost (Supplementary FIG. 8D; r=−0.51, p=0.09), suggesting that BCL2-XL protein (BCL2L1 gene product) expression, on its own, is a poor biomarker for MCL1 inhibitor sensitivity. The lack of correlation between protein and RNA is consistent with what has been described in the literature; this likely reflects the multitude of post-transcriptional and post-translational regulatory influences which contribute to balancing apoptotic protein expression and cell death pathways.

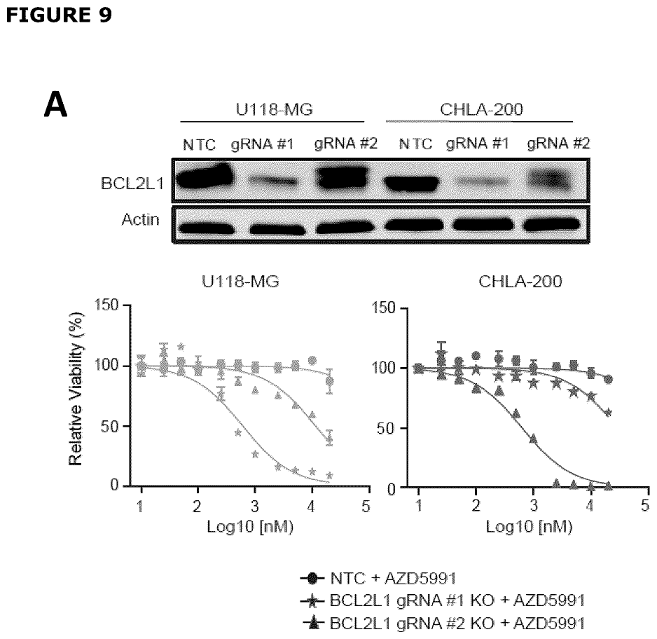

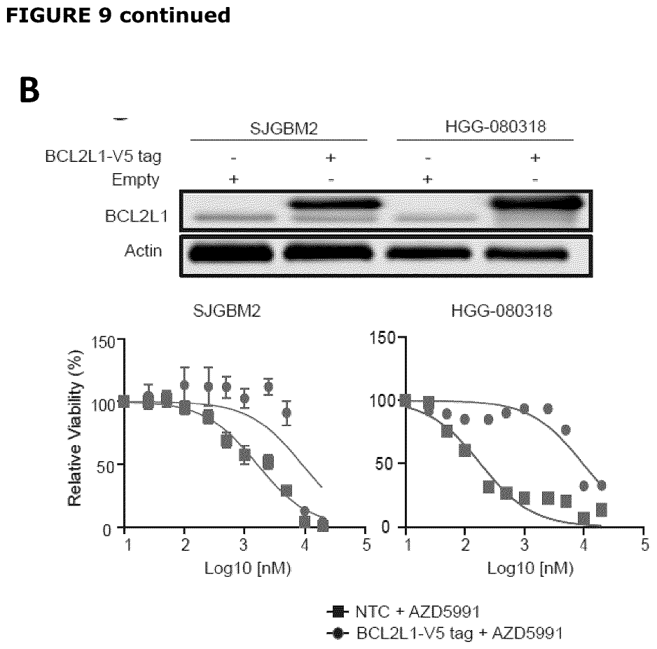

Knockout of BCL2L1 led to a dose dependent sensitization of both adult and paediatric gliomas towards MCL1 inhibitors. Similarly, overexpression of exogenous BCL2L1 led to a reciprocal increase in resistance to MCL1 inhibition pHGG cell lines, confirming the ability for BCL2L1 to compensate for MCL1 loss (FIG. 9A-B).

### Example 4: BCL2L1 Methylation is a Predictor of MCL1 Inhibitor Response in CNS and Non-CNS Paediatric Cancers

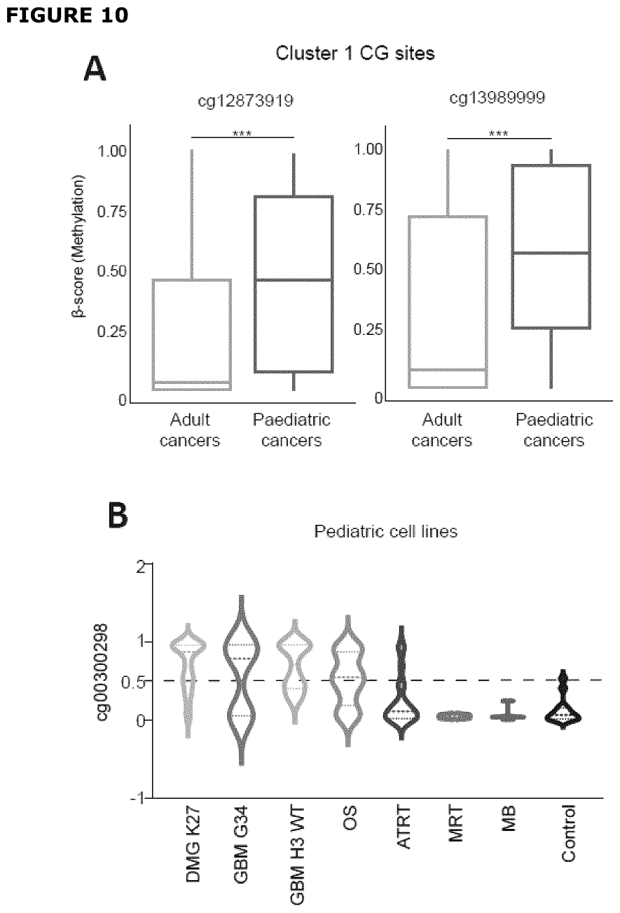

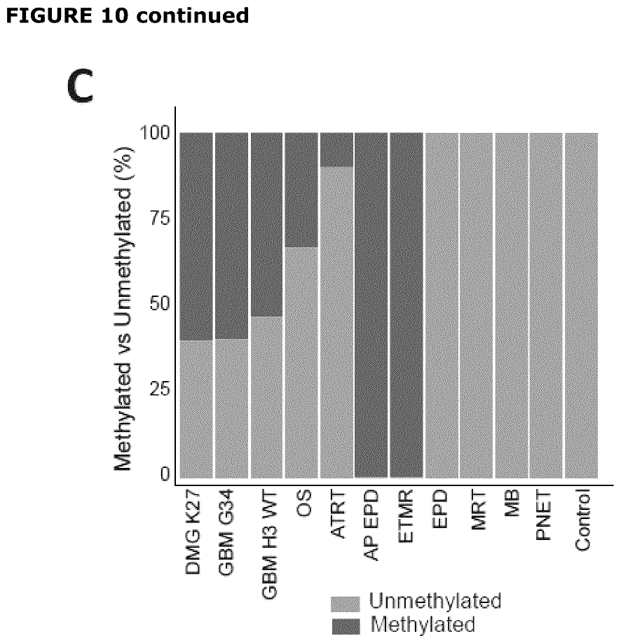

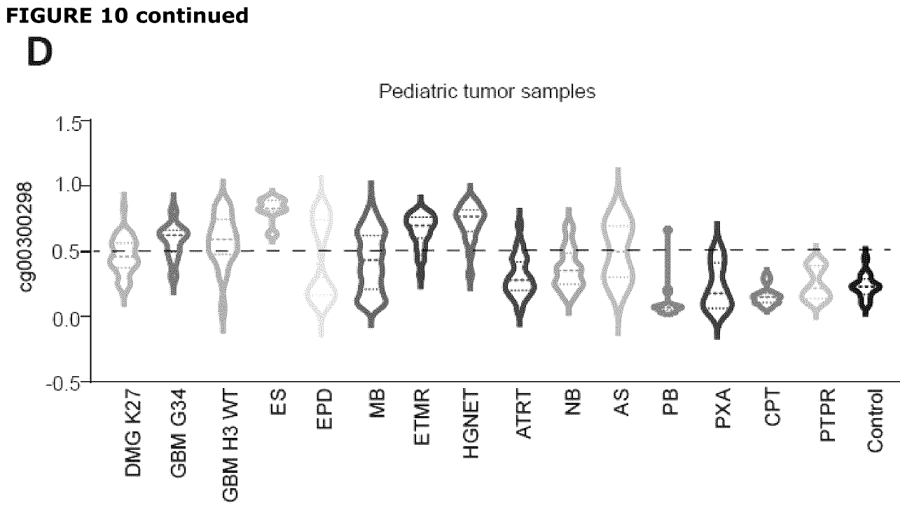

The methylation of cluster 1 CpG sites as a predictive biomarker for MCL1 inhibition was tested in other CNS and non-CNS cancers. Interestingly, analysis of the Cancer Dependency Map (DepMap) cell lines by the Broad Institute identified two CpG sites of cluster 1 i.e. cg12873919 and cg13989999 which were significantly methylated in paediatric cancers compared to its adult counterparts (FIG. 10A; p<0.001).

Evaluation of the Childhood Cancer Model Atlas (CCMA) cell lines revealed a continuum of methylation levels at cg00300298 with Osteosarcoma (OS; n=12), and Atypical Rhabdoid Tumor (ATRT; n=20) showing hypermethylation in a proportion of samples. In contrast, Malignant Rhabdoid Tumor (MRT; n=3), Medulloblastoma (MB; n=7), and non-malignant brain cell lines (Control; n=13) were uniformly hypomethylated at the cg00300298 mark (FIG. 10B). A methylation (0-score) threshold was defined by evaluating AZD5991 response and several cg00300298 methylation cut-offs using Fisher's exact testing (see Table 2 below).

TABLE 2
Fisher's exact test p values for methylation
beta-score cut-offs in cell lines

| Methylated Phenotype | Fisher's Exact |
|---|---|
| Threshold (β-score) | Test (p-value) |
| ≥0.7 | *** |
| ≥0.6 | ** |
| ≥0.5 | * |
| ≥0.4 | ns |

Using a high confidence cg00300298 β-score cut-off of ≥0.7 to define hypermethylation, the cell lines were binned into cg00300298 hypermethylated (β-score>0.7) and hypomethylated (β-score<0.7). cg00300298 hypermethylation was found to be most accentuated in Anaplastic Ependymomas (AP EPD; n=1/1), ETMR (n=1/1) and pHGG cell lines (42/63). In contrast, embryonal-type paediatric tumors including ATRT (18/20), MRT (n=3/3), MB (n=7/7), and Primitive Neuroectodermal Tumor (PNET; n=1/1) were marked by cg00300298 hypomethylation (FIG. 10C). Examination of patient tumor data from DFKZ (Heidelburg) datasets showed lower overall methylation levels compared to cell lines, consistent with low tumor purity of patient specimens which likely have varying proportions of largely hypomethylated non-tumor cells (31). Generally, Ewing sarcoma (ES; n=7), Embryonal Tumors with Multilayered Rosettes (ETMR; n=30), and High-Grade Neuroepithelial Tumor (HGNET; n=21) exhibited higher methylation levels at cg00300298 relative to other cancer types or non-malignant tissue. Conversely, ATRT (n=92), Neuroblastoma (NB; n=38), Astrocytoma (AS; n=2), Pineoblastoma (PB; n=16), Pleomorphic Xanthoastrocytoma (PXA; n=6), Choroid Plexus Tumor (CPT; n=25), Papillary Tumor of the Pineal Region (PTPR; n=13), and non-malignant brain tissue (Control; n=16) were more hypomethylated. Ependymoma (EPD; n=126), Medulloblastoma (MB; n=315), and Astrocytoma (AS; n=2) showed variable methylation across tumors (FIG. 10D).

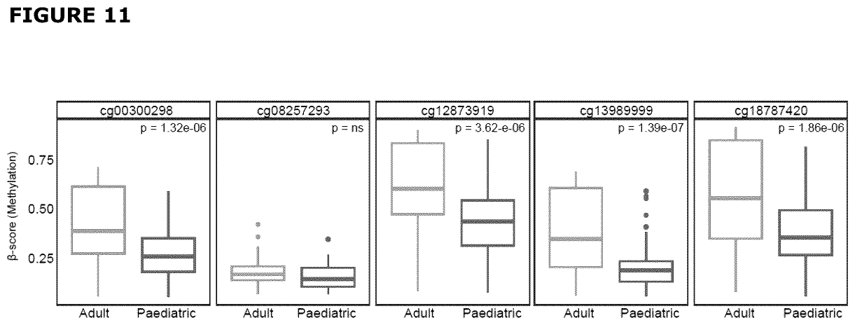

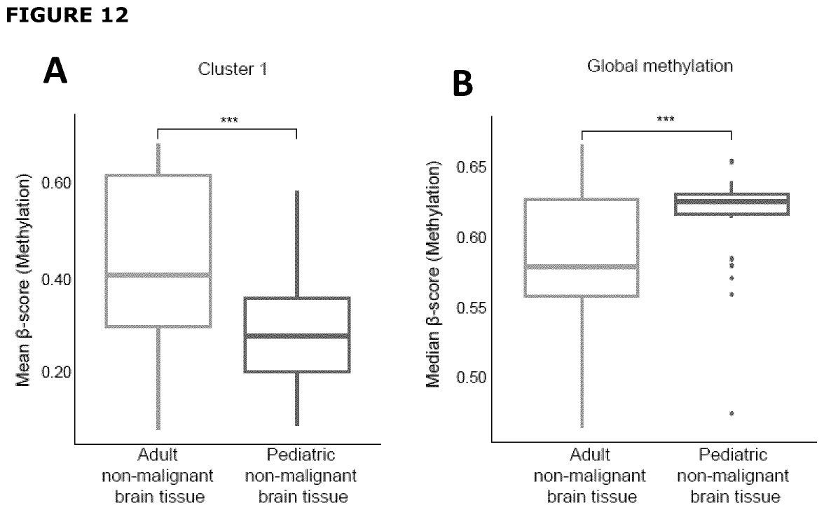

To address whether the BCL2L1 hypermethylation was a byproduct of age-related global DNA hypermethylation, DNA methylation datasets derived from normal brain across a spectrum of ages (13-96) was used to explore the methylation pattern of the cluster 1 CpG sites in non-malignant tissue. There was an age-dependent methylation pattern across various brain regions (cerebral cortex, cerebellum, striatum and hippocampus), with paediatric (n=36) samples exhibiting a significantly hypomethylated phenotype compared to adult (n=182) samples (FIG. 11, FIG. 12A; age-cutoff=21, p<0.0001). However, when examining global methylation patterns, the paediatric population displayed a significantly hypermethylated genome compared to the adult population (FIG. 12B; p<0.0001), highlighting that hypermethylation at cluster 1 CpG sites is a distinctive characteristic of paediatric cancers. These data extend the initial observations in paediatric HGGs and identify other childhood tumor types that harbor hypermethylation at cluster 1 CpG sites that may benefit from MCL1 inhibitor therapy.

### Example 5: BCL2L1 Hypermethylation Predicts MCL1 Dependency In Vivo

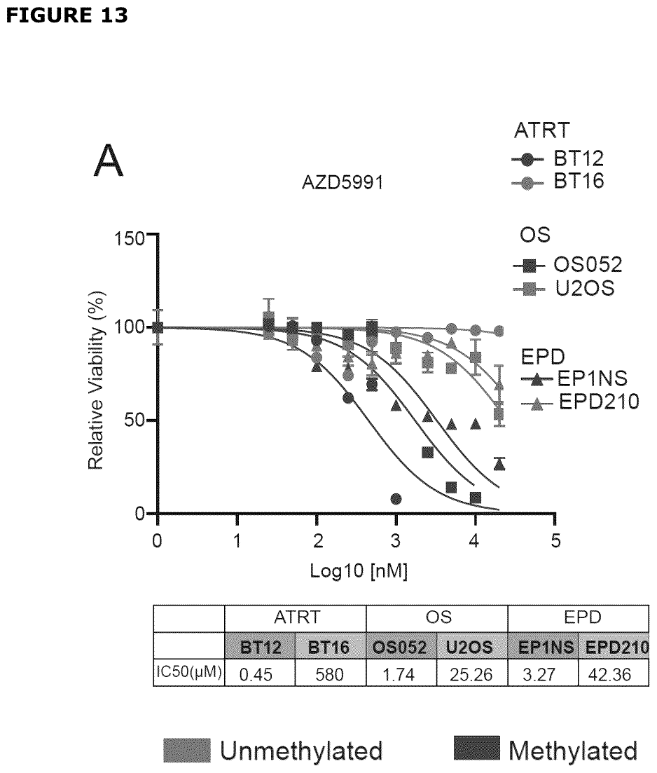

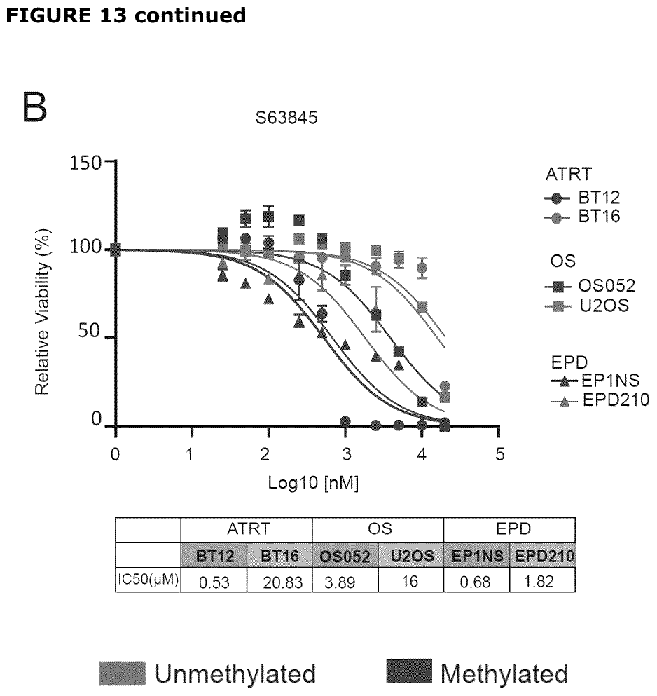

To define whether MCL1 inhibitor therapy in non-pHGG tumors can be defined by the cluster 1 methylation status, AZD5991 and S63845 responses in selected pairs of ATRT, Ependymoma and Osteosarcoma cell lines defined by cluster 1 methylation status were assayed. Consistent with the finding in pHGG, it was found that both MCL1 inhibitors, AZD5991 (FIG. 13A, F test p<0.0001) and S63845 (FIG. 13B; F test p<0.0001) activity correlated to cg00300298 methylation state. For instance, hypermethylated ATRT (BT12) responded at low nanomolar concentrations to both AZD5991 (IC50=425 nM) and S63845 (IC50=528 nM) compared to the matching hypomethylated ATRT line BT16 (IC50>20 μM for both inhibitors), representing a greater than 40-fold increase in sensitivity to MCL1 inhibitors. See Table 3 below.

TABLE 3
BCL2L1 hypermethylation and sensitivity to MCL1 inhibitors

| | ATRT | | OS | | EPD | |
|---|---|---|---|---|---|---|
| IC50 | BT12 | BT16 | OS052 | U2OS | EP1NS | EPD210 |
| (μM) | (Methyl'd) | (Unmethyl'd) | (Methyl'd) | (Unmethyl'd) | (Methyl'd) | (Unmethyl'd) |
| AZD5991 | 0.45 | 580 | 1.74 | 25.26 | 3.27 | 42.36 |
| S63845 | 0.53 | 20.83 | 3.89 | 16 | 0.68 | 1.82 |

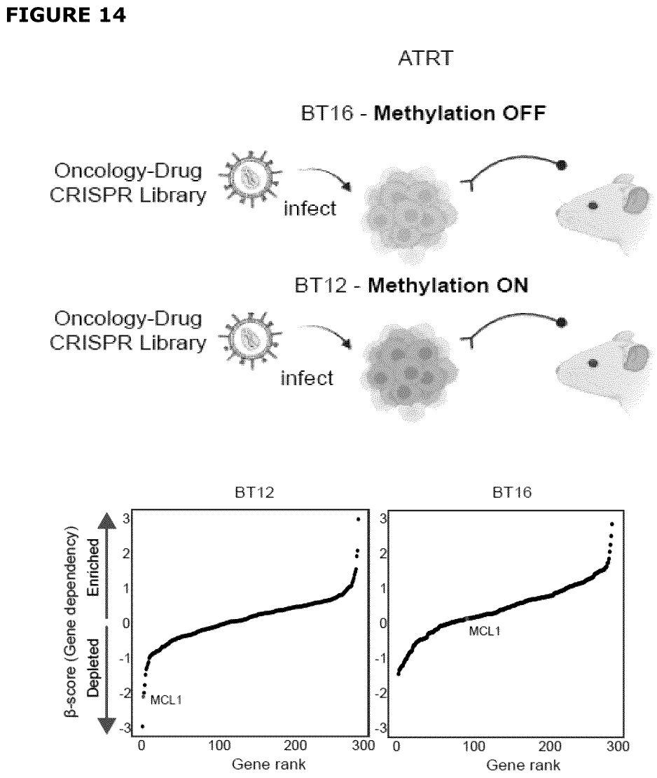

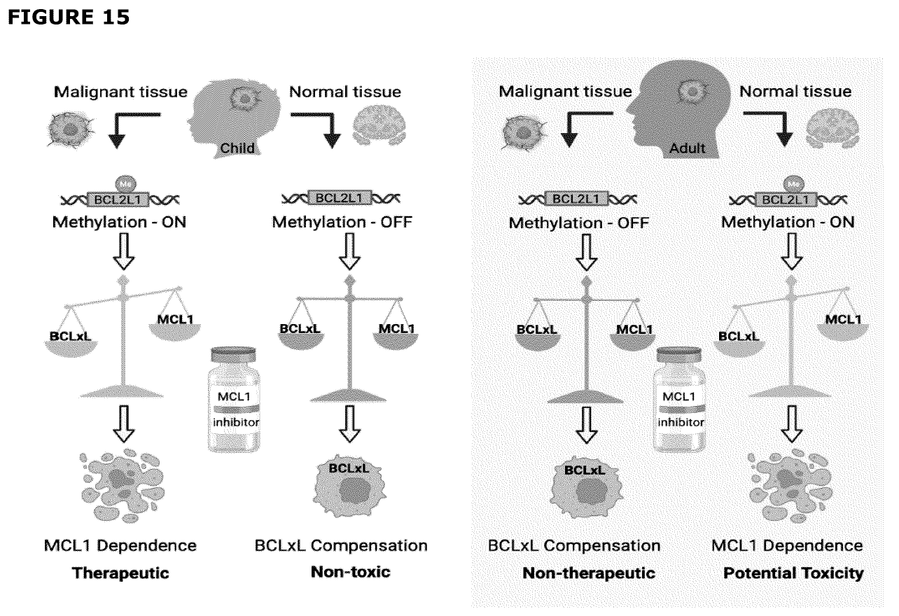

In vivo pooled CRISPR-Cas9 loss-of-function screens was conducted to investigate the dependency of MCL1 in BCL2L1 hypermethylated compared to hypomethylated models. Specifically, ATRT-BT12 (hypermethylated) and ATRT-BT16 (hypomethylated) transduced with a pooled sgRNA library (1666 genes/352 genes, as described above) and implanted intracranially in NSG mice 5 days post transduction (FIG. 14). Consistent with the in vitro data, BT12 (BCL2L1 hypermethylated) was dependent on MCL1 for in vivo growth as compared to the BT16 (BCL2L1 hypomethylated). Remarkably, in BT12 tumors, MCL1 was the highest ranked gene dependency across 290 oncology-focused gene targets and showed essentiality scores on par with positive control ‘common essential genes’ specifically in the presence of BCL2L1 Cluster 1hypermethylation (FIG. 14; BT12=−2.04, BT16=0.21).

These findings demonstrate the use of Cluster 1 methylation as a biomarker of MCL1 dependency and targeted inhibitor response is both broadly applicable and clinically tractable for a diverse set of paediatric tumors.

### Discussion of Results Presented in Examples 1-5

Evasion of apoptosis is a hallmark of cancer and the role of BCL-2 family of proteins in tumor formation and survival is known. For example, BCL2L1 upregulation is a resistance factor of MCL1 inhibitor response in solid tumors and hematological malignancies (Yasuda et al (2020) Cell death & disease 11:1-15); and it has been shown that MCL1 inhibition is effective against a subset of small-cell lung cancer with high MCL1 and low BCL-X L expression. While the results disclosed herein identified a novel association between the ability of BCL2L1 methylation at an exonic-intronic region (N-shore ofCpG island) to predict MCL1 inhibitor response in pHGGs.

Differential DNA methylation at promoter and CpG islands, and other non-island regions such as shores have been previously shown to regulate gene expression in cancer. A correlation between the cg00300298 mark and BCL2L1 expression is described herein.

The BCL-2 family of proteins, similar to many other cellular signaling proteins undergo post-translational modification e.g. phosphorylation, ubiquitination, proteolytic cleavage and proteasomal degradation, which can affect expression/protein enrichment levels. Like epigenetic silencing of BCL2L1, RNA expression exhibited a robust association with MCL1 inhibitor response. However, as methylation of DNA is considered a stable change in DNA, and is less susceptible to treatment-induced alterations compared to gene expression, BCL2L1 methylation at cg00300298 may be a better predictor of MCL1 inhibitor response in pediatric and adolescent cancer, as opposed to relying on protein and RNA expression biomarkers.

TABLE 4
CpG sites in cluster 1 and cluster 2
of the BCL2L1 gene as shown in FIG. 5

| CpG site | Corresponding |
|---|---|
| (Illumina 850k EPIC Methylation | genomic coordinates |
| Array or Illumina 450K | (based on GRCh38/hg38; |
| Methylation Array) | version GCF_000001405.26) |
| cg06892281 | chr20: 31695147-31695148 |
| cg25812375 | chr20: 31700232-31700233 |
| cg17997762 | chr20: 31686061-31686062 |
| cg17168956 | chr20: 31709303-31709304 |
| cg15401244 | chr20: 31680723-31680724 |
| cg14517873 | chr20: 31706215-31706216 |
| cg21948170 | chr20: 31671139-31671140 |
| cg00782854 | chr20: 31723055-31723056 |
| cg12896779 | chr20: 31723263-31723264 |
| cg07379251 | chr20: 31722945-31722946 |
| cg21306641 | chr20: 31722683-31722684 |
| cg03816593 | chr20: 31723900-31723901 |
| cg11551419 | chr20: 31723436-31723437 |
| cg23752198 | chr20: 31724160-31724161 |
| cg11809604 | chr20: 31724044-31724045 |
| cg07602173 | chr20: 31722627-31722628 |
| cg09190106 | chr20: 31722606-31722607 |
| cg04897370 | chr20: 31713376-31713377 |
| cg14002228 | chr20: 31713169-31713170 |
| cg00300298 | chr20: 31721153-31721154 |
| cg08257293 | chr20: 31720637-31720638 |
| cg02538009 | chr20: 31704843-31704844 |
| cg00058652 | chr20: 31720457-31720458 |
| cg11265696 | chr20: 31717824-31717825 |
| cg18787420 | chr20: 31721663-31721664 |
| cg12873919 | chr20: 31721824-31721825 |
| cg13989999 | chr20: 31721914-31721915 |

TABLE 5
Cluster 1 CpG sites as shown in FIG. 5

| CpG site | Corresponding |
|---|---|
| (Illumina 850k EPIC Methylation | genomic coordinates |
| Array or Illumina 450K | (based on GRCh38/hg38; |
| Methylation Array) | version GCF_000001405.26) |
| cg13989999 | chr20: 31721914-31721915 |
| cg12873919 | chr20: 31721824-31721825 |
| cg18787420 | chr20: 31721663-31721664 |
| cg00300298 | chr20: 31721153-31721154 |
| cg08257293 | chr20: 31720637-31720638 |
| cg00058652 | chr20: 31720457-31720458 |
| cg11265696 | chr20: 31717824-31717825 |
| cg04897370 | chr20: 31713376-31713377 |
| cg14002228 | chr20: 31713169-31713170 |
| cg02538009 | chr20: 31704843-31704844 |

1. A method of treating a cancer in a subject in whom the cancer is detected at a paediatric or adolescent stage, the method comprising administering to the subject an agent that inhibits MCL1 activity, wherein the cancer detected in the subject at a paediatric or adolescent stage comprises a cell that exhibits DNA hypermethylation at the BCL2L1 gene locus in comparison to the level of DNA methylation at a BCL2L1 gene locus in a non-cancerous cell.

2. A method of treating a cancer in a subject, the method comprising
a) measuring the level of DNA methylation at the BCL2L1 gene locus of a cancer cell obtained from the subject at a paediatric or adolescent stage;
(b) identifying whether the cancer cell exhibits DNA hypermethylation at the BCL2L1 gene locus in comparison to the level of DNA methylation at the BCL2L1 gene locus of a non-cancerous cell; and
(c) where the subject is identified in step (b) as exhibiting DNA hypermethylation at the BCL2L1 gene locus, administering to the subject a therapeutically effective amount of an agent that inhibits MCL1 activity.

3. A method of stratifying a subject having a cancer to a treatment regimen comprising an agent that inhibits MCL1 activity, the method comprising:
(a) measuring the level of DNA methylation at the BCL2L1 gene locus of a cancer cell obtained from the subject at a paediatric or adolescent stage;
(b) identifying whether the cancer cell exhibits DNA hypermethylation at the BCL2L1 gene locus in comparison to the level of DNA methylation level at the BCL2L1 gene locus of a non-cancerous cell; and
(c) where the subject is identified in step (b) as exhibiting DNA hypermethylation at the BCL2L1 gene locus is stratified to a treatment regimen comprising an agent that inhibits MCL1 activity.

4. (canceled)

5. The method of claim 1, wherein the BCL2L1 gene locus corresponds to the region spanning chr20:31,664,458 to chr20:31,724,161.

6. The method of claim 1, wherein the BCL2L1 gene locus corresponds to the region spanning chr20:31,664,777 to chr20:31,724,161.

7. The method of claim 1, wherein the DNA hypermethylation at the BCL2L1 gene locus is at one or more CpG sites selected from the group consisting of cg06892281, cg25812375, cg17997762, cg17168956, cg15401244, cg14517873, cg21948170, cg00782854, cg12896779, cg07379251, cg21306641, cg03816593, cg11551419, cg23752198, cg11809604, cg07602173, cg09190106, cg04897370, cg14002228, cg00300298, cg08257293, cg02538009, cg00058652, cg11265696, cg18787420, cg12873919 and/or cg13989999.

8. The method of claim 1, wherein the DNA hypermethylation at the BCL2L1 gene locus is at one or more CpG sites within the exon 2 and/or intron 2 junction of BCL2L1 gene.

9. The method of claim 1, wherein the DNA hypermethylation at the BCL2L1 gene locus is at one or more CpG sites within chr20:31,704,843-chr20:31,721,915.

10. The method of claim 1, wherein the DNA hypermethylation at the BCL2L1 gene locus is at one or more CpG sites selected from the group comprising cg13989999, cg12873919, cg18787420, cg00300298, cg08257293, cg00058652, cgl1265696, cg04897370, cg14002228 and cg02538009.

11. The method of claim 1, wherein the DNA hypermethylation at the BCL2L1 gene locus is at one or more CpG sites selected from the group comprising cg00300298, cg08257293, cg12873919, cg13989999 and cg18787420.

12. The method of claim 1, wherein the DNA hypermethylation at the BCL2L1 gene locus is at cg12873919 and/or cg13989999.

13. The method of claim 1, wherein the BCL2L1 gene locus is at cg00300298.

14. The method of claim 1, wherein the cancer is a glioma, an Atypical Teratoid Rhabdoid Tumors (ATRT), an ependymoma (EPD), an Ewing sarcoma (ES), an Embryonal Tumor with Multilayered Rosettes (ETMR), a High-Grade Neuroepithelial Tumor (HGNET) or an osteosarcoma (OS).

15. The method of claim 14, wherein the cancer is a glioma.

16. The method of claim 15, wherein the cancer is a high-grade glioma.

17. The method of claim 1, wherein the non-cancerous cell is a non-cancerous cell from the same subject.

18. The method of claim 1, wherein the agent that inhibits MCL1 activity is an MCL1-siRNA, an MCL-1 antisense oligonucleotide, S64315, S63845, AZD5991, AMG-176, AMG-397, ABBV-475, ANJ810, TTX-180, ABBV-467 and/or PRT1419.

19. The method of claim 1, wherein the agent that inhibits MCL1 activity is AZD5991.

20. The method of claim 1, wherein the cancer is detected in the subject aged between 0-21 years old.

21.-28. (canceled)

29. A kit for analysing a biological sample for the presence of DNA methylation at the BCL2L1 gene locus, according to the methods of claim 1, the kit comprising;
(a) a set of nucleic acid primers capable of amplifying the hypermethylated regions of the BCL2L1 gene locus, and/or
(b.) a set of probes specific for hypermethylated regions of the BCL2L1 gene locus.

30.-40. (canceled)
